(19) United States
(12) Patent Application Publication
(10) Pub. No.: US 2004/0147428 A1
(43) Pub. Date: Jul. 29, 2004

(54) Methods of treatment using an inhibitor of epidermal growth factor receptor

(76) Inventor: John D. Pluenneke, Parkville, MO (US)

Correspondence Address: IMMUNEX CORPORATION, LAW DEPARTMENT, 1201 AMGEN COURT WEST, SEATTLE, WA, 98119, US

(21) Appl. No.: 10/712,122

(22) Filed: Nov. 13, 2003

Related U.S. Application Data
(60) Provisional application No. 60/426,578, filed on Nov. 15, 2002.

(51) Int. Cl. A61K 31/00

(52) U.S. Cl. 514/1

(57) ABSTRACT

The invention provides methods for treating a variety of cell proliferative diseases using an EGFR inhibitor.

This application claims the benefit of U.S. Provisional Application No. 60/426,578, filed Nov. 15, 2002, and which is incorporated herein by reference.

### FIELD OF THE INVENTION

The invention relates to therapeutic treatments for diseases involving inappropriate cell proliferation. More particularly, the invention involves treating such diseases with Epidermal Growth Factor Receptor (EGFR) inhibitors, with or without other therapeutic agents.

### BACKGROUND

EGFR is a receptor tyrosine kinase that normally functions to promote proliferation of cells that express it. EGFR is a transmembrane receptor with an extracellular domain, which binds its various ligands, a transmembrane domain, and a cytoplasmic tyrosine kinase domain. Upon ligand binding, EGFR dimerizes, and its tyrosine kinase domain becomes activated, resulting in transphosphorylation of both receptor subunits. See e.g. Olayioye et al. (2000), EMBO J. 19(13):3159-67. This process, in turn, can activate one or more intracellular signaling cascades, leading to cell proliferation. Hence, inhibition of EGFR can inhibit cell proliferation.

EGFR is expressed and/or overexpressed in a variety of cell proliferative diseases, including cancers. EGFR overexpression is reported to correlate with a high metastatic rate and an increased rate of tumor proliferation. Pavelic et al. (1993), Anticancer Res. 13:1133-38. Thus, EGFR expression may play a role, directly or indirectly, in these disease processes. Various agents that inhibit EGFR have been shown to have anti-tumor activity in certain cancers. However, improvement and new uses of this treatment are possible.

### SUMMARY

The methods of the invention encompass the use of EGFR inhibitors to treat a variety of cell proliferative diseases. Moreover, the use of combinations of EGFR inhibitors with a number of other therapeutic agents to treat cell proliferative diseases are also within the scope of the invention.

The invention provides a method for reducing tumor burden comprising administering to a patient suffering from an EGFR⁺ cancer therapeutically effective amounts of thalidomide and an EGFR inhibitor. The EGFR inhibitor may be a small molecule and may cause diarrhea when used as a single agent.

The invention further provides a method for reducing tumor burden comprising:

(a) administering to a patient suffering from an EGFR⁺ cancer a therapeutically effective dose of an antibody that specifically binds to an antigen expressed on the cancer cells; (b) preparing dendritic cells; (c) combining the dendritic cells with an immunogenic polypeptide, wherein the immunogenic polypeptide is at least nine amino acids long and wherein the immunogenic polypeptide is substantially similar to part or all of EGFR or a tumor-specific variant thereof; and (d) introducing the dendritic cells that have been combined with the immunogenic polypeptide into the patient. In some embodiments, the patient may have primary brain tumor or a brain metastasis, and the antibody may bind specifically to EGFR or to a tumor-specific variant of EGFR. The dendritic cells may be autologous and may be “activated” as described below before introduction into the patient.

In another embodiment, the invention provides a method for reducing tumor burden comprising administering a therapeutically effective amount of an EGFR inhibitor to a human patient suffering from a cancer in which the cancer cells express the CCK_{B}/gastrin receptor and do not overexpress EGFR or express little or no EGFR. The EGFR inhibitor may be a small molecule.

In still another embodiment, the invention provides a method for reducing tumor burden comprising administering to a patient suffering from a hematologic cancer therapeutically effective amounts of an EGFR inhibitor and an antibody that can bind to an antigen that is expressed on the hematologic cancer cells. The EGFR inhibitor may also be an antibody.

Further, the invention provides a method for reducing tumor burden comprising administering to a patient suffering from a hematologic cancer therapeutically effective amounts of an EGFR inhibitor and an anti-neoplastic agent, wherein the anti-neoplastic agent is not a farnesyltransferase inhibitor and wherein the hematologic cancer cells express little or no EGFR. The anti-neoplastic agent may be a chemotherapeutic agent or a non-chemotherapeutic antineoplastic agent.

A further therapeutic method of the invention comprises a method for inhibiting recurrence of gross cystic disease of the breast and/or inhibiting the progression from gross cystic disease of the breast to breast cancer comprising administering to a patient who presently has or has had gross cystic disease of the breast a therapeutically effective amount of an EGFR inhibitor. The EGFR inhibitor may be a small molecule and may be administered orally.

The invention further provides a method for reducing tumor burden comprising administering to a patient suffering from an EGFR⁺ cancer therapeutically effective amounts of a TNF inhibitor and an EGFR inhibitor.

Another embodiment comprises a method for preventing or reducing the frequency or severity of transient ischemic attacks or strokes comprising administering to a patient suffering from cerebral ischemia a therapeutically effective amount of an EGFR inhibitor. The patient may suffer from hypertension or atherosclerosis.

In a further embodiment, the invention provides a method for reducing tumor burden comprising administering to a patient suffering from an EGFR⁺ cancer therapeutically effective amounts of a proteasome inhibitor and an EGFR inhibitor.

In a final embodiment, the invention provides a method for reducing tumor burden comprising administering to a patient suffering from an EGFR⁺ cancer therapeutically effective amounts of a CD40 agonist and an EGFR inhibitor. The cancer cells may express CD40, and the cancer may be an ovarian cancer.

### DETAILED DESCRIPTION

An “activated EGFR” forms multimers, usually homodimers or heterodimers with other erbB receptors, such as, for example, c-erbB-2, transphosphorylates its multimeric partner(s), and activates one or more intracellular signaling cascade(s) that result in cell proliferation. Activation of EGFR occurs, for example, upon ligand binding or by mutation, as occurs in the tumor-specific variants described below.

“Activation” of nuclear factor κB (NF-κB) occurs when inhibitor κB (IκB) is not bound to NF-κB, and NF-κB can therefore localize to the nucleus where it can serve as a transcriptional activator. When IκB is bound to NF-κB, NF-κB is confined to the cytoplasm.

“Anti-neoplastic agents” are chemical agents, compounds, or treatments that have cytotoxic or cytostatic effects on cancer cells. Anti-neoplastic agents may, but need not, be targeted to cells undergoing cell division. Anti-neoplastic agents may, but need not, target cancer cells exclusively or almost exclusively. Anti-neoplastic agents include chemotherapeutic agents, radiation, and chemical agents, compounds, or molecules that are not among the well-known chemotherapeutic agents.

“Autologous,” when used in connection with cells that are taken from and reintroduced into an organism, means that the cells were taken from the same individual organism into which they are reintroduced.

“Cell proliferative disease” means a disease characterized by abnormal cell proliferation which contributes to the disease state.

“Chemotherapy,” as used herein, means the treatment of a cancer patient with a “chemotherapeutic agent” that has cytotoxic or cytostatic effects on cancer cells. A “chemotherapeutic agent” specifically targets cells engaged in cell division and not cells that are not engaged in cell division. Chemotherapeutic agents directly interfere with processes that are intimately tied to cell division such as, for example, DNA replication, RNA synthesis, protein synthesis, the assembly, disassembly, or function of the mitotic spindle, and/or the synthesis or stability of molecules that play a role in these processes, such as nucleotides or amino acids. A chemotherapeutic agent therefore has cytotoxic or cytostatic effects on both cancer cells and other cells that are engaged in cell division. Chemotherapeutic agents are well-known in the art and include, for example: alkylating agents (e.g. busulfan, temozolomide, cyclophosphamide, lomustine (CCNU), methyllomustine, streptozotocin, cis-diamminedi-chloroplatinum, aziridinylbenzo-quinone, and thiotepa); inorganic ions (e.g. cisplatin and carboplatin); nitrogen mustards (e.g. melphalan hydrochloride, ifosfamide, chlorambucil, and mechlorethamine HCl); nitrosoureas (e.g. carmustine (BCNU)); anti-neoplastic antibiotics (e.g. adriamycin (doxorubicin), daunomycin, mitomycin C, daunorubicin, idarubicin, mithramycin, and bleomycin); plant derivatives (e.g. vincristine, vinblastine, vinorelbine, paclitaxel, docetaxel, vindesine, VP-16, and VM-26); antimetabolites (e.g. methotrexate with or without leucovorin, 5-fluorouracil with or without leucovorin, 5-fluorodeoxyuridine, 6-mercaptopurine, 6-thioguanine, cytarabine, 5-azacytidine, hydroxyurea, deoxycoformycin, gemcitabine, and fludarabine); podophyllotoxins (e.g. etoposide, irinotecan, and topotecan); as well as actinomycin D, dacarbazine (DTIC), mAMSA, procarbazine, hexamethylmelamine, pentamethylmelamine, L-asparaginase, and mitoxantrone, among many known in the art. See e.g. Cancer: Principles and Practice of Oncology, 4^{th} Edition, DeVita et al., eds., J.B. Lippincott Co., Phildelphia, Pa. (1993). Alkylating agents and nitrogen mustard act by alkylating DNA, which restricts uncoiling and replication of strands. Methotrexate, cytarabine, 6-mercaptopurine, 5-fluorouracil, and gemcitabine interfere with nucleotide synthesis. Plant derivatives such a paclitaxel and vinblastine are mitotic spindle poisons. The podophyllotoxins inhibit topoisomerases, thus interfering with DNA replication. Antibiotics doxorubicin, bleomycin, and mitomycin interfere with DNA synthesis by intercalating between the bases of DNA (inhibiting uncoiling), causing strand breakage, and alkylating DNA, respectively. Other mechanisms of action include carbamoylation of amino acids (lomustine, carmustine), and depletion of asparagine pools (asparaginase). Merck Manual of Diagnosis and Therapy, 17^{th} Edition, Section 11, Hematology and Oncology, 144. Principles of Cancer Therapy, Table 144-2 (1999). Specifically included among chemotherapeutic agents are those that directly affect the same cellular processes that are directly affected by the chemotherapeutic agents listed above.

“Dendritic cells” (DCs) are a heterogeneous population of cells with distinctive cell morphology that display the cell surface proteins CD1 a, CD4, CD86, or HLADR. U.S. Pat. No. 6,017,527. DCs originate from bone marrow cells, peripheral blood progenitors, and/or mononuclear cells that express CD34 on their cell surface. Flt-3 ligand (Flt3-L), granulocyte macrophage stimulating factor (GM-CSF), interleukin 4 (IL-4), tumor necrosis factor α (TNFα), interleukin 3 (IL-3), c-kit ligand, fusions of GM-CSF and IL-3, and/or combinations these proteins can be used to enhance the generation and/or maturation of DCs. U.S. Pat. No. 6,017,527. For example, DC maturation and/or generation can be enhanced in at least several ways: (1) in vivo by administering Flt3-L; (2) in vitro by combining GM-CSF plus interleukin 4 (IL-4) with blood monocytes; and/or (3) in vitro by combining Flt3-L, alone or plus GM-CSF, with hematopoietic progenitor cells from bone marrow or umbilical cord blood. DCs can be activated using the following agents: CD40 agonists, such as CD40 ligand (CD40L) (U.S. Pat. No. 6,017,527); lipopolysaccharides (Labeur et al. (1999), J. Immunol. 162:168-75); TNFα; GM-CSF; monocyte conditioned medium (Romani et al. (1996), J. Immunol. Methods 196(2):137-51 and Bender et al. (1996), J. Immunol. Methods 196(2):121-35); or combinations of these agents. Activation of DCs allows them to effectively present antigens to T killer cells so as to stimulate T killer cells to kill cells that display these antigens in the context of MHC class I.

“EGFR⁺ cancers” include cancers in which the cancer cells express EGFR or a tumor-specific EGFR variant, such as EGFRvI, EGFRvII, EGFRvIII, etc. See e.g. Kuan et al. (2001), Endocrine-Related Cancer 8:83-96.

“EGFR inhibitors” include any molecule that can affect the biological activity of EGFR, for example, by interfering with (1) the interaction of EGFR with its ligands, (2) the dimerization of EGFR, (3) the activation by EGFR of signaling pathway(s), (4) the tyrosine kinase activity of EGFR, and/or (5) the phosphorylation of EGFR. Preferably, an EGFR inhibitor preferentially inhibits EGFR over other tyrosine kinase molecules.

“EGFR inhibitor proteins” are proteins such as those described herein that can inhibit EGFR, as well as any proteins developed in the future that can inhibit EGFR.

“Humanized,” when it refers to an antibody, means that the antibody is wholly or partially the product of expression from a gene (or genes) that is (are) of human origin.

As used herein, any of the following assay results, among others, would indicate that an “immune response against EGFR” has been mounted: 1) antibodies that bind specifically to EGFR and/or to a tumor-specific variant thereof are produced in quantities detectable in blood by, for example, ELISA assay; 2) following injection with a protein comprising part or all of EGFR, a tumor-specific variant thereof, or a substantially similar protein, cytotoxic T lymphocytes isolated from an organism are capable of lysing cells expressing EGFR or a tumor-specific variant thereof, whereas cytotoxic T lymphocytes isolated from the same organism before injection could not lyse such cells; or 3) following injection with a protein comprising part or all of EGFR, a tumor-specific variant thereof, or a substantially similar protein, a group of animals is statistically more likely to be resistant to tumor formation caused by EGFR⁺ cancer cells than are animals that have not been injected with such a protein.

“MHC class I molecules” are polypeptides expressed mainly on the cell surface of most cell types. MHC class I molecules have the capacity to associate with potentially antigenic fragments of proteins, including those expressed by the cell and those phagocytosed by the cell, and to display these fragments on the cell surface. The display of an antigen in the context of an MHC class I molecules on an activated DC can induce activation of a T killer cell, enabling the activated T killer cells to kill cells displaying the antigen in the MHC class I context.

“MHC class II molecules” are proteins expressed mainly on cells of the immune system including B cells, activated T cells, and DCs, among others. Paul, The Immune System: An Introduction, in Fundamental Immunology, Paul, ed., 3-19, Raven Press, New York, 1989. MHC class II molecules have the capacity to associate with potentially antigenic fragments of proteins, which can be larger than the fragments displayed by MHC class I molecules, and to display these fragments on the surface of the cell. The display of a fragment in the context of MHC class II by a DC can stimulate a T helper cell that expresses a T cell receptor that recognizes the antigen in the MHC class II context to secret CD40L, thereby activating DCs.

“Natural killer cells” are immune system cells that can kill antibody-coated cells. Paul, supra.

“Non-chemotherapeutic anti-neoplastic agents” are chemical agents, compounds, or molecules having cytotoxic or cytostatic effects on cancer cells other than chemotherapeutic agents. Non-chemotherapeutic antineoplastic agents may, however, be targeted to interact directly with molecules that indirectly affect cell division such as cell surface receptors, including receptors for hormones or growth factors. However, non-chemotherapeutic antineoplastic agents do not interfere directly with processes that are intimately linked to cell division such as, for example, DNA replication, RNA synthesis, protein synthesis, or mitotic spindle function, assembly, or dissassembly. Examples of non-chemotherapeutic anti-neoplastic agents include inhibitors of Bcl2, inhibitors of farnesyltransferase, anti-estrogenic agents such as tamoxifen, anti-androgenic compounds, interferon, arsenic, retinoic acid, retinoic acid derivatives, antibodies targeted to tumor-specific antigens, and inhibitors of the Bcr-Abl tyrosine kinase (e.g. the small molecule STI-571 marketed under the trade name GLEEVEC™ by Novartis, N.Y. and N.J., USA and Basel, Switzerland), among many possible non-chemotherapeutic anti-neoplastic agents.

A “protein” is a polypeptide comprising at least 9 amino acids, optionally at least 20, at least 30, at least 40, at least 50, at least 75, at least 100, or at least 200 amino acids.

“Small molecules,” as used herein, encompasses molecules other than proteins or nucleic acids without strict regard to size. When EGFR inhibitors that are “small molecules” (or “small molecule EGFR inhibitors”) are referred to, what is meant is non-protein, non-nucleic acid inhibitors of EGFR. In many cases, such inhibitors are smaller than EGFR inhibitors that are proteins or nucleic acids.

For the purposes of the invention, two proteins or nucleic acids are “substantially similar” if they are at least 80%, optionally at least 90%, 95%, 97%, 99%, or 99.5% identical to each other in amino acid or nucleotide sequence and maintain or alter in a desirable manner a biological activity of the unaltered protein. Examples of amino acid substitutions that are conservative substitutions, unlikely to affect biological activity, including the following: Ala for Ser, Val for Ile, Asp for Glu, Thr for Ser, Ala for Gly, Ala for Thr, Ser for Asn, Ala for Val, Ser for Gly, Tyr for Phe, Ala for Pro, Lys for Arg, Asp for Asn, Leu for Ile, Leu for Val, Ala for Glu, Asp for Gly, and these changes in the reverse. See e.g. Neurath et al., The Proteins, Academic Press, New York (1979). The percent identity of two amino acid or two nucleic acid sequences can be determined by visual inspection and mathematical calculation, or more preferably, the comparison is done by comparing sequence information using a computer program. An exemplary computer program is the Genetics Computer Group (GCG; Madison, Wis.) Wis. package version 10.0 program, ‘GAP’ (Devereux et al., 1984, Nucl. Acids Res. 12:387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, (Nucl. Acids Res. 14:6745, 1986, as described by Schwartz and Dayhoff, eds., Atlas of Polypeptide Sequence and Structure, National Biomedical Research Foundation, pp. 353-358, 1979) or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used.

“T killer cells” are cells present in the blood that express CD8 on their cell surface and, when activated, have the capacity to kill cells that express antigens in the context of MHC class I that are recognized by T cell receptors on the cell surface of the T killer cell.

“Tumor burden” refers to the number of viable cancer cells, the number of tumor sites, and/or the size of the tumor(s) in a patient suffering from a cancer. A reduction in tumor burden can be observed, for example, as a reduction in the amount of a tumor-associated antigen or protein in a patient's blood or urine, a reduction in the number of tumor cells or tumor sites, and/or a reduction in the size of one or more tumors.

The invention is directed towards methods for treating patients suffering from a cell proliferative disease with EGFR inhibitors or combinations including EGFR inhibitors plus other therapeutic agents. Preferably, the patient is a human, but the subject methods may be applied to any mammal, including domestic animals such as pets and farm animals. Also provided are compositions for use in such methods that include an effective amount of an EGFR inhibitor and, in some cases, an effective amount of another therapeutic agent, plus a suitable diluent, excipient, or carrier.

Cell proliferative disesases, including cancer, involve the unregulated and/or inappropriate proliferation of cells, sometimes accompanied by destruction of adjacent tissue and growth of new blood vessels, which can allow invasion of cancer cells into new areas, i.e. metastasis. Included within conditions treatable with the invention described herein are non-malignant conditions that involve inappropriate cell growth, including colorectal polyps, cerebral ischemia, gross cystic disease, polycystic kidney disease, benign prostatic hyperplasia, and endometriosis. Other cell proliferative diseases that can be treated using the methods of the present invention are cancers in which the cancer cells can express EGFR, including osteosarcomas, glioblastomas, gliomas, melanomas, and meningiomas, and lung, breast, head and neck, bladder, ovarian, skin, prostate, cervical, gastric, renal cell, pancreatic, colorectal, endometrial, and esophageal cancers, EGFR⁺ cancers that commonly metastasize to bone, and tumors that express gastrin. As used herein, “gastrointestinal cancer” encompasses gastric, esophageal, pancreatic, and colorectal cancers. Also treatable using the methods of the invention are hematologic cancers and cancers that express gastrin, but do not overexpress EGFR or may express little or no EGFR.

Due to the complex and interrelated molecular controls regulating cell growth and apoptosis and to spontaneously-occurring drug resistant cancer cells, treating cancer with multiple therapeutic agents simultaneously may be beneficial. Multiple inhibitors of different cell-signaling pathways could be advantageous in treating cancer, since, for example, activation of a pathway promoting apoptosis may not override molecular signals promoting cell growth. As another example, inflammation and cancer have long been associated, and inflammatory cytokines and chemokines may contribute to malignant progression. Weitzman and Gordon (1990), Blood 76(4):655-63; Balkwill and Mantovani (2001), Lancet 357:539-45. Thus, the inhibition of different signaling pathways leading to inflammation and/or cell growth and/or the activation of pathways leading to apoptosis may all be effective cancer treatments, which are more effective when multiple pathways, rather than a single pathway, are targeted by the treatment. In addition, a combination therapy may be more effective than a therapy using only one therapeutic agent because of the possible existence of cancer cells resistant to at least one, but not all, of the therapeutic agents used in the combination therapy.

EGFR is a transmembrane receptor tyrosine kinase that is activated by ligand binding. EGFR, which is also called c-erbB-1, belongs to the type I family of growth factor receptors, which also includes c-erbB-2 (HER2), c-erbB-3, and c-erbB-4. Gullick and Srinivasan (1998), Breast Cancer Res. Treat. 52(1-3):43-53. The mature human EGFR protein contains 1186 amino acids comprising an extracellular domain including amino acids 1 through 621, a transmembrane domain including amino acids 621 through 644, and a cytoplasmic domain including amino acids 645 through 1186. Kuan et al. (2001), Endocrine-Related Cancer 8:83-96; Ullrich et al. (1984), Nature 309:418-25. Twenty four predominantly hydrophobic amino acids are encoded in the EGFR cDNA immediately upstream of sequence encoding the amino terminus of the mature protein. These are likely a signal peptide that facilitates the transfer of the nascent peptide into the lumen of the endoplasmic reticulum. Ullrich et al., supra. The extracellular domain comprises the ligand binding site(s), and the cytoplasmic domain comprises a region with tyrosine kinase activity and a C-terminal tail comprising tyrosines that can be phosphorylated. Binding of one of the ligands described below causes dimerization (or oligomerization) and activation of the tyrosine kinase domain, which mediates transphosphorylation of both receptor subunits. Mendelsohn and Lippman, Principles of Molecular Cell Biology of Cancer: Growth Factors, in Cancer: Principles and Practice of Oncology, DeVita et al., eds., pp. 114-133, 120. The dimers can be homodimers or heterodimers comprising two different members of the c-erbB receptor family. Olayioye et al. (2000), EMBO J. 19(13):3159-67. Heterodimers can elicit different intracellular signals than homodimers. Moreover, the identity of the ligand can determine whether a homodimer or a particular heterodimer is formed. Olayioye et al., supra. Once a ligand has bound to EGFR, both are taken inside the cell where they may be catabolized in lysosomes. Alternatively, EGFR may be recycled by return to the cell surface. Mendelsohn and Lippman, supra at 118.

EGFR activation can activate single or multiple intracellular signaling cascades, and activation can result from a variety of stimuli. EGFR normally can activate a number of signaling cascades upon activation in response to ligand binding, including the Ras/extracellular signal-related (ERK) pathway, which activates members of the mitogen-activated protein (MAP) kinase family, and phosphoinositol 3-kinase, which can activate numerous pathways that, in general, promote cell growth and inhibit apoptosis. Moscatello et al. (1998), J. Biol. Chem. 273(1):200-206; Antonyak et al. (1998), J. Biol. Chem. 273(5):2817-2822; Katso et al. (2001), Ann. Rev. Cell Dev. Biol. 17:615-75. In addition, it has been shown that the activation of G protein coupled receptors by ligand binding can lead to EGFR activation due to increased processing of the EGFR ligand heparin binding EGF (hb-EGF) by metalloproteases. Gschwind et al. (2001), Oncogene 20(13): 1594-1600. Moreover, activation of EGFR by many diverse agents other than its ligands has also been demonstrated. Hirota et al. (2001), J. Biol. Chem. 276(28):25953-58.

EGFR has multiple ligands, the binding of which can activate EGFR. Among these are epidermal growth factor (EGF), hb-EGF, transforming growth factor α (TGFa), amphiregulin, and hepatocyte growth factor, among others. Mendelsohn and Lippman, Principles of Molecular Cell Biology of Cancer: Growth Factors, in Cancer: Principles and Practice of Oncology, DeVita et al., eds., pp.114-133, 120; Miyazaki et al. (1999), Gastroenterology 116:78-89. TGFa is produced by many tumor cell lines, and unregulated expression of TGFa can result in cancer in at least one model system. Mendelsohn and Lippman, supra at 120. Some EGFR ligands have been reported to have functions that may or may not be related to their activation of EGFR, such as regulating the production of transcription factors for the gastrin gene. Watson et al. (2000), Int. J. Cancer 87:20-28.

EGFR is not expressed in most tissue types at detectable levels, but is expressed in skin and placenta. As used herein, overexpression of EGFR by cancer cells is expression that clearly exceeds the levels of expression found in vivo in most normal cells of the same tissue type from which the cancer cells arose. For example, the expression of EGFR by renal cancer cells can be compared to the expression of EGFR by normal kidney cells. One of skill in the art will appreciate that absolute levels of EGFR expression in renal cancer cells that are overexpressing EGFR may be less than absolute levels seen in some normal cells that express high levels of EGFR, such as placenta cells.

Expression of EGFR can be detected at the RNA or the protein level. At the RNA level, EGFR expression can be detected by Northern blot, reverse transcription, reverse transcription followed by polymerase chain reaction (PCR), in situ hybridization, in situ hybridization accompanied or preceded by PCR, reverse transcription and labeling of RNA followed by hybridization to a nucleic acid array, among many possible techniques. Expression of EGFR at the protein level can be detected, for example, by the following methods, among many possible techniques: immune staining of whole cells (see e.g. Pavelic et al. (1993); Anticancer Res. 13:1133-38); Western blot; and pre-purification by affinity chromatography followed by total protein quantitation by methods such as measurement of absorbance at 280 nanometers or a Lowry assay, among many other possible techniques.

A preferred method of measuring the expression of EGFR is by antibody staining with an antibody that binds specifically to EGFR employing a labeling strategy that makes use of luminescence or fluorescence. Such staining may be carried out on fixed tissue or cells that are ultimately viewed and analyzed under a microscope. Staining carried out in this manner can be scored visually or by using optical density measurements. Staining may also be carried out using either live or fixed whole cells in solution. Such cells can be analyzed using a fluorescence activated cell sorter (FACS), which can determine both the number of cells stained and the intensity of the luminescence or fluorescence. Such techniques are well known in the art, and exemplary techniques are described in Luwor et al. ((2001), Cancer Res. 61:5355-61). One of skill in the art will realize that other techniques of detecting EGFR expression might be more or less sensitive than these techniques. As meant herein, cancer cells express little or no EGFR if little or no EGFR can be detected using an antibody staining technique that relies on luminescence or fluorescence.

EGFR Inhibitors

EGFR inhibitors include a number of classes of molecules including proteins, nucleic acids, and various classes of small molecules, including carbohydrates or their analogues. Among the small molecules are those that inhibit the interaction between EGFR and at least one of its ligands and those that inhibit the tyrosine kinase activity of EGFR. Among the proteins are antibodies or antigen-binding fragments thereof that can inhibit the interaction of EGFR with at least one of its ligands, optionally with all of its ligands. Such antibodies can bind to EGFR, a tumor-specific version of EGFR, or one or more of its ligands. Such antibodies may or may not be human or “humanized,” may be single chain or multi-chain, and may be monoclonal or polyclonal. Also included are antibodies that can bind to EGFR and that are conjugated to radioactive, luminescent, cytotoxic, or differentiation-inducing compounds. Anti-idiotypic antibodies against antibodies against EGFR that can elicit an immune response against EGFR are also included. Also among the proteins are transcriptional repressors of EGFR or its ligands and ligand-binding polypeptides, such as polypeptides comprising all or part of a protein identical or similar to the extracellular region of EGFR. Furthermore, the proteins include proteins that can be used as vaccines to elicit an immune response against EGFR or one of its ligands. Such an immune response may interfere with ligand binding to EGFR or may cause the killing of cells that display part or all of EGFR or a tumor-specific variant of EGFR on its cell surface. Also included are proteins that can bind to EGFR and can thereby prevent its activation or proteins conjugated to cytotoxins that can bind to EGFR. Both D- and L-form amino acids can be included in any of these proteins. Among the nucleic acids are the following: antisense nucleic acids; ribozymes and DNAs that encode them; triple helix-forming nucleic acids; interfering RNAs and DNAs that encode them (see e.g. Fjose et al. (2001), Biotechnol. Ann. Rev. 7:31-57) that can inhibit the expression of EGFR or its ligands; and nucleic acids that can be used as vaccines to elicit an immune response against EGFR or its ligands. Other EGFR inhibitors encompassed by the invention may act to preferentially inhibit proliferation of cells expressing or overexpressing EGFR or a tumor-specific variant thereof by an unknown mechanism. An example of such an inhibitor is VRCTC-310, which comprises a purified fraction from the venom of the South American rattlesnake, Crotalus durissus terrificus. See Donato et al. (1996), Biochem. Pharmacol. 51(11): 1535-43. Other EGFR inhibitors contemplated include the bifunctional molecules described in US Patent Application No. 20020045570, which are reported to have the effect of promoting the selective degradation of tyrosine kinases in the c-erbB family.

In one embodiment, the anti-EGFR antibody is a partially or fully human antibody generated by procedures that involve immunizing a non-human transgenic animal with part or all of EGFR or a tumor-specific variant thereof. For example, the transgenic animal may be a transgenic mouse, into which genetic material encoding one or more human immunoglobulin polypeptide chains has been introduced. Monoclonal antibodies can be produced by conventional procedures, e.g. by immortalizing spleen cells harvested from the transgenic animal after completion of the immunization schedule. The spleen cells can be fused with myeloma cells to produce hybridomas using conventional procedures, and hybridoma cell lines producing the desired monoclonal antibody can be identified.

The present invention contemplates the use of the following anti-EGFR antibodies, antigen-binding fragments of these antibodies, and substantially similar proteins, among others: human antibodies produced by hybridoma lines generated by procedures involving immunization of transgenic mice, including ABX-EGF (Abgenix), which is an antibody produced by the hybridoma cell line E7.6.3 described in U.S. Pat. No. 6,235,883 B1 and WO 98/50433; the human antibodies described in US Patent Application No. 2002/0058033; the human and murine antibodies described in WO 95/20045; the C225 antibody (ImClone Systems, Inc., New York, N.Y., USA) and a humanized version thereof; the humanized anti-EGFR antibodies described in US Patent Application No. 2002/0065398; the chimeric antibody MDX-447 (Medarex Inc. of Annandale N.J., USA; see Belt et al. (1999), American Society of Clinical Oncology, Program/Proceedings, Abstract #1667); the monoclonal antibodies described in U.S. Pat. Nos. 5,459,061, 5,470,571, 5,558,864, 5,891,996, and U.S. Pat. No. 6,217,866 B1; the anti-idiotypic antibodies of EP Patent No. 0 745 612 and U.S. Pat. No. 5,969,107; antibody conjugates with radioactive, luminescent, cytotoxic, or differentiation-inducing compounds, such as those described in EP Patent No. 0 659 439, U.S. Pat. No. 5,470,571, and US Application 20020001587; single-chain anti-EGFR antibodies and multi-chain antibodies described in U.S. Pat. No. 5,844,093 and US Patent Application No. 2002/0058033; antibodies against a tumor-specific version of EGFR described in WO 91/16350 and U.S. Pat. No. 5,212,290 and U.S. Pat. No. 5,981,725; and single and multi-chain antibodies and toxin conjugates of such antibodies described in U.S. Pat. No. 6,129,915.

The invention also encompasses use of the proteins (and nucleic acids that encode them) described immediately below, and proteins (and nucleic acids) substantially similar to these, as EGFR inhibitors. All or part of proteins GCF1 and GCF2 (described in WO 97/41226 and references cited therein), which can act as transcriptional repressors of EGFR, are EGFR inhibitors as understood herein. Fusions of all or part of EGF with all or part of the diptheria toxin (such as those described in described U.S. Pat. No. 5,614,488 and Shaw et al. (1991), J. Biol. Chem. 266(31):21118-24) are EGFR inhibitors as understood herein. The cytotoxic action of such EGFR inhibitors results from binding to EGFR, subsequently becoming internalized by the cell, and, once internalized, inhibiting protein synthesis by virtue of the ADP-ribosylation of elongation factor 2 leading to inhibition of protein synthesis and cell death. Chloroquine can lessen or eliminate the negative effects of such an EGF fusion with all or part of diptheria toxin on protein synthesis. Shaw et al., supra. The cyclic peptides described in U.S. Pat. No. 5,183,805 that bind to EGFR (and may be conjugated to all or part of diptheria toxin or a monoclonal antibody) are included among proteins considered EGFR inhibitors herein. Such cyclic peptides can stimulate or inhibit cell proliferation of cells expressing EGFR, depending on dose, and, when complexed to all or part of diptheria toxin, may be cytotoxic. Proteins useful as vaccines to elicit an immune response against EGFR, such as those described in U.S. Pat. No. 5,894,018 and U.S. Pat. No. 6,224,868 B1 are also EGFR inhibitors as understood herein. These include polypeptides comprising part or all of EGFR or a tumor-specific variant thereof, e.g. EGFRvIII, which is described below, or substantially similar proteins. Such proteins may stimulate the production of antibodies that recognize one or more epitopes on EGFR or a tumor-specific variant thereof and thus promote killing of cells that display these molecules. Such proteins may also stimulate the proliferation of killer T cells whose receptors specifically bind to an epitope on EGFR or a tumor-specific variant thereof. A purified fraction of the venom from the South American rattlesnake, Crotalus durissus terrificus, comprising crotoxin and cardiotoxin known as VRCTC-310 (Ventech Research) is also a contemplated EGFR inhibitor. This agent, which is also a potent neurotoxin, can preferentially inhibit the proliferation of cells expressing EGFR by an unknown mechanism that may or may not be related to the phospholipase A2 activity of the fraction. Donato et al. (1996), Biochem. Pharmacol. 51(11):1535-43; Costa et al. (1998), American Society of Clinical Oncology, Program/Proceedings, Abstract #1696; Costa et al. (1997), American Society of Clinical Oncology, 1997 Program/Proceedings, Abstract #820).

Proteins (such as those described in WO 93/22339 and U.S. Pat. No. 5,874,528) that bind to EGFR ligands are also contemplated EGFR inhibitors. The EGFR-inhibiting action of such peptides may be due to their binding of ligands, which may make the ligands less available to bind and activate EGFR on the surface of cells expressing it. Such proteins can comprise sequences substantially similar to all or part of EGFR or a tumor-specific variant thereof and can include a full length EGFR or tumor-specific EGFR variant sequence or the extracellular domain of EGFR or a fragment of the extracellular domain that retains the ability to bind at least one EGFR ligand. Such forms of EGFR can bind to at least one EGFR ligand, including EGF, TGFa, and/or hb-EGF, among others. In some embodiments, such an inhibitor may be an oligomer comprising two or more proteins, each of which comprises protein sequences substantially similar to all or part of EGFR and can bind at least one EGFR ligand. Such proteins comprising protein sequences substantially similar to all or part of EGFR can be fusion proteins comprising a region that promotes oligomerization (as described elsewhere herein) such as, for example, a leucine zipper or an Fc region of an antibody. Such ligand-binding EGFR inhibitors can also be heterodimers comprising all or part of EGFR and all or part of another protein. The other protein can be another c-erbB receptor, such as, for example c-erbB2 (HER2).

EGFR inhibitors contemplated by the invention further include any polypeptide that can bind specifically to EGFR described herein, including antibodies or other proteins, fused to a toxic substance. Such toxic substances include bacterial toxins, such as diptherotoxin, Pseudomonas exotoxin, or tetanus toxin, or ribosome inactivating proteins, such as gelonin, saporin, ricin, or the mistletoe lectins described in US Patent Application No. 20020045208, maytansine (described in U.S. Pat. No. 3,896,111), maytansinoids (described in U.S. Pat. No. 4,151,042), or maytansinol or maytansinol analogues (described in U.S. Pat. Nos. 4,137,230, 4,248,870, 4,256,746, 4,260,608, 4,265,814, 4,294,757, 4,307,016, 4,308,268, 4,308,269, 4,309,428, 4,313,946, 4,315,929, 4,317,821, 4,322,348, 4,331,598, 4,361,650, 4,364,866, 4,424,219, 4,450,254, 4,362,663, and U.S. Pat. No. 4,371,533), among others.

Soluble forms of EGFR and other proteins described above that can inhibit EGFR can also be fused to other molecules, including polypeptides, to enhance effectiveness or in vivo half life or to facilitate purification. Among polypeptides that may be fused to such proteins are the following: multimerization domains, including various portions of antibody-derived polypeptides (including an Fc portion of an antibody) as described, e.g., by Ashkenazi et al. (Proc. Natl. Acad. Sci. 88:10535 (1991)), Byrn et al. (Nature 344:677 (1990)), and Hollenbaugh and Aruffo (“Construction of Immunoglobulin Fusion Polypeptides,” in Current Protocols in Immunology, Suppl. 4, pages 10.19.1-10.19.11 (1992)) and leucine zippers as described by Landschulz et al. (Science 240:1759 (1988)). Other polypeptides that may be fused to EGFR inhibitor proteins include protein tags such as polyarginine, polyhistidine, or HATTM (Clontech), which is a naturally-occurring sequence of non-adjacent histidine residues that possess a high affinity for immobilized metal ions, the antigenic identification peptides described in U.S. Pat. No. 5,011,912 and in Hopp et al. (Bio/Technology 6:1204 (1988)), and others as described in Sassenfeld (TIBTECH 8:88-93 (1990)), Brewer et al. (in Purification and Analysis of Recombinant Proteins, pp. 239-266, Seetharam and Sharma (eds.), Marcel Dekker, Inc. (1991)), and Brewer and Sassenfeld (in Protein Purification Applications, pp. 91-111, Harris and Angal (eds.), Press, Inc., Oxford England (1990)). Soluble forms of any EGFR inhibitor that is a protein, as well as any other therapeutic protein described herein, can be fused to non-protein molecules that enhance in vivo half life or activity, such as polyethylene glycol or various sugar or carbohydrate moieties, among others.

The present invention contemplates the use of the following nucleic acids, or substantially similar nucleic acids, as EGFR inhibitors: nucleic acids encoding any of the EGFR inhibitor proteins described above; nucleic acids that inhibit the expression of EGFR, including antisense nucleic acids, ribozymes, DNA enzymes (such as those described in U.S. Pat. No. 5,914,269, 6,057,156, and U.S. Pat. No. 6,187,585), triple helix-forming nucleic acids, and/or interfering RNAs (see e.g. Fjose et al. (2001), Biotechnol. Ann. Rev. 7:31-57; Plasterk (2002), Science 296:1263-65); and nucleic acids capable of inducing an immune response against EGFR described in U.S. Pat. No. 6,127,344.

The present invention contemplates the use of the following small molecules, among others: the tyrosine kinase inhibitors ZD-1839 (known as IRESSA™, AstraZeneca), BIBX-1382 (Boehringer Ingleheim), OLX-103 (Merck & Co., Whitehouse Station, N.J., USA), EKB, EKB-569 and EKI-785 (Wyeth Ayerst Research, Pearl River N.Y., USA; Torrance et al. (2000), Nature Medicine 6(8):1024-28), CGP 59326A (Novartis Pharma AG, Basel, Switzerland; Lydon et al. (1998), Int. J. Cancer 76(1):154-63), OSI-774 (trade name TARCEVA™, OSI Pharamceuticals), CI-1033 (an irreversible EGFR inhibitor), and those described in WO 00/56703, WO 96/40648, WO 95/24190, WO 95/20045, WO 96/30347, WO 96/33980, and WO 96/07657 and U.S. Pat. Nos. 5,760,041, 5,773,476, and U.S. Pat. No. 6,057,320; and the carbohydrates and carbohydrate analogues described in U.S. Pat. No. 6,281,202,B1 that bind to EGFR and inhibit its kinase activity in response to EGF.

Also included within the scope of EGFR inhibitors embraced by the invention are the dendritic cells (DCs) described below that are pulsed with part or all of a protein substantially similar to part or all of EGFR-or a tumor specific variant thereof as described in Heimberger et al. ((2002), Neurosurgery 50(1): 158-64).

### Indications and Treatments

EGFR⁺Cancers

As mentioned above, EGFR can be overexpressed in a number of cancers, and this overexpression sometimes correlates with poor prognosis. These conditions include osteosarcoma, glioblastoma, melanoma, meningioma, glioma, and lung, breast, head and neck, bladder, ovarian, skin, prostate, cervical, renal cell (i.e., kidney), gastrointestinal (including pancreatic, gastric, colorectal, and esophageal), and endometrial cancers. Mendelsohn and Lippman, Principles of Molecular Cell Biology of Cancer: Growth Factors, in Cancer: Principles and Practice of Oncology, Devita et al., eds., pp.114-133, 121, J.B. Lippincott Company, 1993; Nicholson et al. (2001), Eur. J. Cancer 37 Suppl. 4:S9-15; Moscatello et al. (1998), J. Biol. Chem. 273(1):200-206; Kuan et al. (2001), Endocrine-Related Cancer 8:83-96. Some EGFR inhibitors have demonstrated anticancer effects. Other molecules can also affect disease progression of cancers that express EGFR (EGFR⁺ cancers). As discussed above, combination treatments can be more effective than therapies using a single therapeutic agent.

EGFR Inhibitor Plus COX-2 Inhibitor

The invention provides a method for reducing tumor burden comprising administering effective amounts of an EGFR inhibitor and a Cyclooxygenase-2 (COX-2) inhibitor to a patient suffering from an EGFR⁺ cancer. This method can be used to treat a patient that has received, will receive, and/or is receiving chemotherapy and/or radiation treatment. Further, treatment with chemotherapy and/or radiation can continue or resume after treatment with the COX-2 inhibitor and the EGFR inhibitor.

COX-2 contributes to inhibition of apoptosis, increased angiogenesis, increased invasiveness, modulation of inflammatory and immune responses, and conversion of procarcinogens to carcinogens. COX-2 inhibitors that are appropriate include selective COX-2 inhibitors, which inhibit the COX-2 enzyme without a substantial inhibition of the COX-1 enzyme, and non-selective COX-2 inhibitors. Among appropriate COX-2 inhibitors are: sulindac (Wyeth-Ayerst Research); 4-[5-(4-methylphenyl)-3-(trifluoromethyl)-1H-pyrazol-1-yl] benzenesulfonamide (also called celecoxib or CELEBREX®) as described in WO 99/30721; N-(2-cyclohexyloxy-4-nitrophenyl)methanesulfonamide (also called NS 398; Cayman Chemical, Ann Arbor, Mich., USA); 3-(phenyl)-4-(4-(methylsulfonyl)phenyl)-2-(5H)-furanone (also called NK-0966 or VIOXX® and described as compound (63) in WO 99/30721); acetylsalicylic acid; ibuprofen; piroxicam; and the COX-2 inhibitors described in WO 99/30721 and US Patent Application 2001/0041726, among others. When a COX-2 inhibitor is used in combination with an EGFR inhibitor, appropriate doses of either or both may be lower than doses required when either is used as a single agent. For example, celecoxib as a single agent may be used at dosages from about 10 mg to about 1000 mg, optionally from about 100 mg to about 600 mg, or from about 200 mg to about 400 mg. Doses can be given at intervals of four times, thrice, twice, or once per day or at intervals of every other day, or once every three, four, five, six, or seven days.

EGFR Inhibitor Plus Tumor Necrosis Factor Inhibitor

The present invention provides a method for reducing tumor burden comprising administering to a human patient suffering from an EGFR⁺ cancer therapeutically effective amounts of a tumor necrosis factor (TNF) inhibitor and an EGFR inhibitor. The effects of TNF are mediated through its receptors, which are described in U.S. Pat. No. 5,395,760 and U.S. Pat. No. 5,610,279. TNF, although it does exhibit antitumor activity toward some cancer cell lines in vitro, promotes angiogenesis, promotes bone resorption through its promotion of osteoclast formation, and induces phosphorylation and internalization of EGFR, all of which are likely to contribute to the progression of a cancer. Hirota et al (2001), J. Biol. Chem. 276(28):25953-58; Osaku et al (2001), Oncology Reports 8(4):855; Leek et al (2000), J. Pathol. 190:430-36; Sedlak et al (1994), Exp. Chemotherapy 40:51-56; Azuma et al. (2000), J. Biol. Chem. 275(7):48584868. Moreover, TNF can increase expression of EGFR in at least two kinds of carcinoma cells (Sedlak et al. (1994), Chemotherapy 40:51-56; Osaku et al. (2001), Oncology Reports 8(4):855-60). Thus, it is contemplated herein that TNF inhibitors can simultaneously increase the effectiveness of EGFR inhibitors by decreasing the expression of EGFR and exert other anticancer effects such as blocking angiogenesis and/or bone resorption.

Examples of suitable TNF inhibitors include: TNFR:Fc, which is a dimer of two molecules of the extracellular portion of the p75 TNFα receptor, each molecule consisting of a 235 amino acid polypeptide that is fused to a 232 amino acid Fc portion of human IgG₁ (etanercept), and which is sold by Immunex Corporation (Seattle, Wash., USA) under the trade name ENBREL®; other polypeptides comprising a soluble portion of TNFR that can bind TNF; the TNF receptors and binding proteins described in US Patent Application No. 2002/0090676; the antibodies adalimumab D2E7 or afelimomab (Abbott), which neutralize or inhibit TNF; the humanized antibody infliximab, sold under the tradename REMICADE™ (Centocor); ISIS 104838, an antisense inhibitor of TNF; the humanized antibodies CDP571 and cA2 described in EP Patent No. 0 516 785 B1 and U.S. Pat. No. 5,656,272, respectively; other anti-TNF antibodies including CDP 571 (Celltech) and those described in U.S. Pat. No. 5,994,510 and EP Patent No. 0 492 448 A1; the peptides described in U.S. Pat. No. 5,795,859 and U.S. Pat. No. 6,107,273; the polypeptides described in US Patent Application No. 2002/0091243 that promote enzymatic cleavage and release of TNF receptors from a cell surface; small molecules such as pentoxifylline or thalidomide; inhibitors of the TNFα converting enzyme such as those described in U.S. Pat. No. 5,594,106; and the TNF inhibitors described in U.S. Pat. No. 6,143,866. Suitable doses of TNFR:Fc (ENBREL®; as a single agent, range from 0.5 mg to 150 mg, optionally from 20 mg to 60 mg, or from 25 mg to 50 mg. When a TNF inhibitor and an EGFR inhibitor are used in combination, suitable doses of either or both may be somewhat lower than doses that are appropriate for either as a single agent. One of skill in the art will realize that the selected dosage can depend on the frequency of administration. For example, it may be appropriate to administer 50 mg once a week or, alternatively, 25 mg twice per week. Suitable doses for infliximab (REMICADE™) can range from 0.1 mg/kg to 50 mg/kg, preferably from 1 mg/kg to 15 mg/kg. Suitable doses for afelimomab can range from 0.1 mg/kg to 50 mg/kg, preferably from 0.3 mg/kg to 3.0 mg/kg.

EGFR Inhibitor Plus an Antiestrogen Compound

The present invention provides a method for reducing tumor burden comprising administering therapeutically effective amounts of an EGFR inhibitor and an antiestrogen compound to a patient suffering from an EGFR⁺ cancer. In some embodiments, the EGFR⁺ cancer may not be breast cancer. An estrogen receptor can be expressed on the cancer cells. Suitable antiestrogen compounds include, but are not limited to, (Z)₂-[4-(1,2-diphenyl-1-butenyl) phenoxy]-N,N-dimethylethanamine 2-hydroxy-1,2,3-propanetricarboxylate (1:1) (also known as tamoxifen), which is currently used to treat breast cancer. Tamoxifen has been reported to inhibit the proliferation of cancer cells in which proliferation increases in response to estradiol and the proliferation of cancer cells that do not respond to estradiol and that may or may not express either estrogen receptor (α or β). Lee et al. (2000), Oncogene 19(33):3766-73; Caracta et al. (2001), American Thoracic Society, 97^{th} International Conference, San Francisco, Calif., Poster D108. Thus, anti-estrogen compounds may act through plural pathways to inhibit the proliferation of some cancer cells, and not all of these pathways necessarily include an estrogen receptor. Caracta et al., supra.

EGFR Inhibitor Plus a Proteasome Inhibitor

Proteasomes may play a role in the progression of cancer, and proteasome inhibitors have anticancer activity. See e.g. Adams et al. (1999), Cancer Res. 59:2515-22; Cusack et al. (2001), Cancer Res. 61:3535-40. Proteasomes are large, multimeric proteases that exist in all eukaryotic cells and serve the function of degrading ubiquitinated proteins. Many proteins degraded by this pathway serve important regulatory roles in cell growth. Inhibition of their timely degradation can interfere with cell division, thus potentially having anticancer effects. For example, proteasomes are responsible for the degradation of inhibitor protein κB (IκB), which inhibits nuclear factor κB (NF-κB) by confining this transcription factor to the cytoplasm. Hirota et al. (2001), J. Biol. Chem. 276(28):25953-58. NF-κB mediates the transcription of at least two factors involved in cell adhesion, i.e., vascular cell adhesion molecule-1 and intercellular adhesion molecule-1, both of which can play a role in the progression of a cancer in vivo. Adams et al. (1999), Cancer Res. 59:2615-22. Other data also suggests the involvement of NF-κB in tumorigenesis at least in some systems. Biswas et al. (2001), Proc. Natl. Acad. Sci. USA 98(18):10386-91; Adams et al. supra. The transmission of the mitogenic signal mediated by an activated EGFR leads to activation of NF-κB. Biswas et al. (2000), Proc. Natl. Acad. Sci. 97(15):8542-47. Thus, inhibition of proteasomes and of EGFR both tend to confine NF-κB to the cytoplasm, although probably by distinct molecular mechanisms.

The invention provides a method for reducing tumor burden comprising administering to a human patient suffering from an EGFR⁺ cancer therapeutically effective amounts of a proteasome inhibitor and an EGFR inhibitor. Appropriate proteasome inhibitors can include antisense molecules, ribozymes, triple helix-forming nucleic acids, DNA enzymes, or interfering RNAs (see e.g. Fjose et al. (2001), Biotechnol; Ann. Rev. 7:31-57) that inhibit the expression of proteins necessary for necessary for the degradation of appropriate proteins by proteasomes, including proteins that form the proteasome or ubiquitin-activating enzymes such as 47885 (described in US Patent Application 2002/0039773), among others. Another appropriate proteasome inhibitor is pyrazylCONH(CHPhe)CONH(Chisobutyl)B-(OH)₂ (also called PS-341), which is currently in clinical trials as a cancer treatment (Adams (2001), Semin. Oncol. 28(6):613-19). Other molecules that inhibit proteasomes and having substantially similar selectivity of action, would also be appropriate. See Adams et al. (1999), Cancer Res. 59:2615-2622. Suitable doses of PS-341, as single agent, range from about 0.01 mg/m to 1.6 mg/m², optionally from about 0.1 mg/m² to about 1.4 mg/m². See Papandreou et al. (2001), Amer. Soc. Clin. Oncol., Program/Proceedings, Abstract # 340. When a proteasome inhibitor and an EGFR inhibitor are used in combination, appropriate doses for either or both may be lower than when either is used as a single agent.

EGFR Inhibitor Plus Thalidomide

Loss of appetite and progressive weight loss are common symptoms among patients with advanced cancer. Jatoi and Loprinzi (2001), Curr. Opin. Clin. Nutr. Metab. Care 4(3): 179-82. This problem may be worsened by treatment with small molecule EGFR inhibitors that can cause gut toxicity, including diarrhea, which is a dose-limiting side effect in some cases. Ciardiello and Tortora (2001), Clin. Cancer Res. 7:2958.

The present invention provides method for reducing tumor burden comprising administering to a human patient suffering from an EGFR⁺ cancer therapeutically effective amounts of thalidomide and an EGFR inhibitor. This method can be particularly appropriate when the EGFR inhibitor is a small molecule and/or another kind of EGFR inhibitor that causes gut toxicity.

Thalidomide has pleitropic biological effects. Among these are inhibition of angiogenesis, anti-tumor activity, inhibition of the synthesis of tumor necrosis factor α (TNFα), and constipation. Folkman (2001), Semin. Oncol. 28(6):536-42; Davis and Dickerson (2001), Am. J. Hosp. Palliat. Care 18(5):347-51; Rajkumar (2001), Oncology (Huntingt) 15(7):867-74; Govindarajan et al. (2000), Lancet 356:566-67. Thalidomide is currently used for the treatment of leprosy. Physicians' Desk Reference, Medical Economics Co., Inc., Montvale, N.J., USA (2002). Thalidomide has also shown promising anti-tumor activity for myeloma, AIDS-related Kaposi's sarcoma, renal cell carcinoma, and glioblastoma multiforme. See e.g. Adlard (2000), Anticancer Drugs 11(10):787-91. Thalidomide can ameliorate diarrhea caused by a chemotherapeutic agent, irinotecan. Govindarajan et al., supra. The combination of an EGFR inhibitor and thalidomide is especially appropriate when the EGFR inhibitor causes gut toxicity when used as a single agent therapy, but it is also appropriate to counteract the weight loss commonly seen in cancer patients that is not caused by treatment with an EGFR inhibitor. This treatment can provide multiple, simultaneous lines of attack upon the cancer while minimizing gastrointestinal distress, especially diarrhea.

Thalidomide can be administered in daily doses from about 5 mg to about 1200 mg, optionally from about 50 mg to about 700 mg or from about 100 mg to about 600 mg. One of skill in the art can adjust the frequency of administration and the dose based on the nature of the condition and response of the patient, realizing that different doses of thalidomide and/or an EGFR inhibitor may or may not be appropriate when a combination, rather than a single agent, is used.

EGFR Inhibitor Plus Keratinocyte Growth Factor

The present invention further provides a method for reducing tumor burden comprising administering therapeutically effective amounts of an EGFR inhibitor and a keratinocyte growth factor to a patient suffering from an EGFR⁺ cancer, thereby minimizing mucositis and/or skin toxicity, and/or gut toxicity, including diarrhea, that may be caused by the disease or by the EGFR inhibitor. The keratinocyte growth factor (KGF) family consists of KGF-1 and KGF-2. KGF-1 and −2 are expressed by stromal cells and act as paracrine mediators of proliferation of epithelial cells, including skin cells and epithelial cells lining the gastrointestinal system, including mouth, throat, esophagus, stomach, and intestinal cells. Finch et al. (1989), Science 245:752-55; Igarishi et al. (1998), J. Biol. Chem. 273:13230-35; WO 96/25422. Skin reactions such as an acneiform rash are associated with at least some EGFR inhibitors. See e.g. Figlin et al. (2001), Amer. Soc. Clinical Oncol., Program/Proceedings, Abstract #1102. Human KGF-1 produced from recombinant cells has been shown to reduce mucositis among patients receiving total body irradiation or high dose chemotherapy. Spielberger et al. (2001), Amer. Soc. Clin. Oncol., Program/Proceedings, v. 20: Abstract 25; see also Danilenko (1999), Toxicol. Pathol. 27(1):64-71; Xia et al. (1999), J. Pathol. 188(4):431-38.

EGFR Inhibitor Plus TRAIL

The TNF Related Apoptosis Inducing Ligand (TRAIL) protein can induce apoptosis in various target cells, including cancer cells and cells infected with a virus, and is described in detail in WO 97/01633. The present invention provides a method for reducing tumor burden comprising administering therapeutically effective amounts of an EGFR inhibitor and a TRAIL protein, which may be a biologically active fragment, variant, or oligomer that comprises an amino acid sequence at least 20 or, optionally, at least 30 amino acids long that is at least 80% identical to SEQ ID NO:2 or SEQ ID NO:6 of WO 97/01633, to a patient suffering from an EGFR⁺ cancer. Included in TRAIL proteins contemplated by the invention are all the TRAIL polypeptides, including soluble TRAIL polypeptides, fusions, fragments, and oligomers, as described in WO 97/01633. Biological activity for TRAIL proteins includes the ability to induce apoptosis in at least some cancer cell types. Apoptosis may be assayed in any appropriate manner, including the DNA laddering and cell killing assays described in WO 97/01633, Examples 5, 8, and 9.

EGFR⁺ Cancers that Metastasize to Bone

Bone is the third most common site of metastasis after lung and liver and is a major site of metastasis in three very common cancers that can be EGFR⁺ cancers, that is, breast, prostate, and lung cancers. Mundy (1997), Cancer 8(Suppl.):1546-56. Other cancers that can be EGFR⁺ cancers can also metastasize to bone, including kidney and gastrointestinal cancers, as well as melanomas. Bone metastasis often signals a dramatic decrease in quality of life for a patient because of accompanying intractable pain, impaired mobility, hypercalcemia, bone fractures, and spinal cord or nerve root compression. See e.g. Woodhouse et al. (1997), Cancer 8 (Suppl):1529-37; Coleman (1997), Cancer 8 (Suppl):1588-94. Prevention, inhibition, and/or treatment of metastasis to bone may be particularly important in cancers in which patients commonly survive for long periods after diagnosis of cancer. In this context, a long period can mean a period of one or more months or years.

Metastasis, which is the spread of a cancer from an original tumor site to a distant site, encompasses several distinct steps. The generation of blood vessels allows the primary tumor to grow and increases the chances that tumor cells can reach the bloodstream and colonize new sites. Cell adhesion also plays an important role in metastasis since tumor cells must both detach and attach to cells and/or extracellular matrices to successfully metastasize. Translocation of cancer cells across extracellular matrices, such as bone or collagenous tissue, requires at least some destruction of this tissue. Finally, tumor growth at a new site requires cancer cell proliferation. Woodhouse, supra.

Remodeling of bone occurs throughout adult life and is characterized by a balanced action of osteoclasts, which resorb bone, and osteoblasts, which form bone. Bone metastasis usually involves an imbalance between the processes of bone growth and bone resorption, usually being characterized by increased bone resorption caused by increased osteoclast activity or, less frequently, by abnormal bone growth due to increased osteoblast activity. Mundy, supra. Receptor activator of NF-κB (RANK) is a cell surface receptor that is expressed on the surface of osteoclast precursors and is described in U.S. Pat. No. 6,017,729. Upon binding of receptor activator of NF-κB ligand (RANKL, which is also called osteoprotegerin ligand (OPGL), osteoclast differentiation factor (ODF), or TNF-related activation-induced cytokine (TRANCE)), RANK stimulates the formation of osteoclasts (osteoclastogenesis), which causes bone resorption. RANKL is expressed on the surface of bone marrow stromal cells (BMSCs) and osteoblasts in response to bone-resorbing signals. Yasuda et al. (1998), Proc. Natl. Acad. Sci. 95:3597-3602. Co-culture of either one of these cell types with osteoclast precursor cells is sufficient to induce the formation of osteoclast-like cells. Moreover, RANKL alone is sufficient to induce osteoclast formation in osteoclast precursors in cell culture. These data strongly suggest that BMSCs and/or osteoblasts play a role in osteoclastogenesis through their expression of RANKL. Yasuda et al., supra. It is contemplated herein that since BMSCs express EGFR, EGFR inhibitors may inhibit growth of BMSCs, thereby inhibiting osteoclastogenesis.

Provided herein is a method for treating, preventing, or inhibiting bone metastasis of an EGFR⁺ cancer comprising administering to a human patient suffering from an EGFR⁺ cancer therapeutically effective amounts of a RANKL inhibitor plus an EGFR inhibitor. EGFR inhibitors can inhibit the growth of EGFR⁺ cancer cells directly and can inhibit BMSC proliferation, since BMSCs express EGFR. Since BMSCs may contribute to bone metastasis through their role in osteoclastogenesis, inhibition of their growth may also inhibit bone metastasis. RANKL inhibitors can interfere with the interaction between RANK and RANKL, likely inhibiting the creation of osteoclasts and preventing bone resorption. Thus, osteoclastogenesis can be attacked on two different levels by this treatment: 1) growth of cells expressing RANKL, including BMSCs, is inhibited; and 2) RANKL is directly inhibited.

Examples of RANKL inhibitors include: (1) antibodies that bind to either RANK or RANKL and thereby block binding of RANKL by RANK without activating RANK and without stimulating osteoclast formation; (2) polypeptides other than antibodies that can block binding of RANKL by RANK; (3) small molecules that block binding of RANKL by RANK; (4) a polypeptide comprising a RANK polypeptide or a substantially similar protein that is capable of binding RANKL; and (5) a ribozyme, an antisense, an interfering RNA, a DNA enzyme, a triple helix forming polynucleotide, or an interfering RNA that can inhibit expression of RANK and/or RANKL, among others. Examples of RANK polypeptides include RANK/Fc fusion proteins and dimers containing two RANK/Fc fusion proteins as described in U.S. Pat. No. 6,017,729. RANK/Fc fusion proteins comprise a RANK polypeptide, preferably a soluble RANK polypeptide, and an Fc polypeptide derived from the Fc region of an antibody.

RANKL inhibitors include, but are not limited to: a human RANK polypeptide; a polypeptide comprising a fragment of a human RANK polypeptide (preferably a soluble fragment), wherein the fragment is capable of binding specifically to RANKL; a polypeptide comprising an amino acid sequence that is at least 90% identical to the amino acid sequence of a native human RANK protein, wherein said polypeptide is capable of binding specifically to RANKL; a soluble polypeptide comprising an amino acid sequence that is at least 90% identical (or at least 95% identical) to the extracellular domain of a native human RANK protein, wherein said soluble polypeptide is capable of binding RANKL; a polypeptide comprising all or part of a human osteoprotegrin binding protein (OPG, as described in U.S. Pat. No. 6,316,408 B1), or a fragment thereof, which can specifically bind to RANKL; a polypeptide comprising a sequence at least 90% identical to all or part of the sequence of human OPG protein that can specifically bind to RANKL; and a soluble fusion polypeptide comprising a soluble portion of human OPG, or a substantially similar protein, fused to an Fc domain of a human IgG1 antibody that can specifically bind to RANKL, such as that described in U.S. Pat. No. 6,316,408 B1.

In particular embodiments, the RANK polypeptide is in oligomeric form. Such oligomers may comprise two or more soluble human RANK polypeptides, for example. In particular embodiments, the oligomer comprises two or more fusion proteins, wherein each fusion protein comprises a soluble human RANK polypeptide and a second polypeptide that promotes oligomerization, such as one of the oligomerization-promoting polypeptides described elsewhere herein.

As discussed above, soluble (secreted) forms of RANK may be employed in the methods provided herein. Soluble forms of transmembrane receptor proteins typically lack the transmembrane region that would cause retention of the protein on the cell surface. In one embodiment of the invention, a soluble RANK polypeptide comprises the extracellular domain of the protein, or a fragment of the extracellular domain that retains the ability to bind RANKL. A soluble RANK polypeptide may include the cytoplasmic domain, or a portion thereof, as long as the polypeptide is secreted from the cell in which it is produced.

RANKL inhibitors can be administered at any effective dosage and frequency until best response is achieved. For example, RANK/Fc dosage can be in the range from 0.01 mg to 500 mg, optionally from 10 mg to 100 mg or from 25 mg to 50 mg, if dosage given does not depend on body weight or skin area. Alternatively, dosage may be calculated in terms of body weight. If so, appropriate doses can range from 0.01 mg/kg to 70 mg/kg, optionally from about 0.01 mg/kg to about 20 mg/kg or from about 0.1 mg/kg to about 5 mg/kg. One of skill in the art can adjust the frequency of administration and the dose based on the nature of the condition and response of the patient, realizing that lower or higher doses of a RANKL inhibitor and/or an EGFR inhibitor may be appropriate when a combination, rather than a single agent, is used.

Treatment of EGFR⁺ Cancers with Combination Therapies Comprising an Antibody and Pulsed Dendritic Cells

Some cancers express tumor-specific versions of the EGFR gene that have deletions or rearrangements. See U.S. Pat. No. 5,981,725 and Kuan et al. (2001), Endocrine-Related Cancer 8:83-96. Such alterations have been correlated with gene amplification of EGFR and poor prognosis. Kuan et al., supra. The most common rearranged version of EGFR is EGFRvIII, which is expressed in more than 50% of glioblastoma multiforme cases that exhibit EGFR gene amplification. Kuan et al., supra. Other tumor-specific versions of EGFR have also been described. Kuan et al., supra. EGFRvIII has also been reported in breast, ovarian, prostate, and lung carcinomas. EGFRvIII bears a deletion that is entirely within the extracellular domain of EGFR from amino acid 6 through 273. EGFRvIII, like some, but not all, other tumor-specific versions of EGFR, is constitutively activated, that is, it does not require ligand binding to stimulate cell proliferation. Kuan et al., supra. Transfection of NIH3T3 fibroblast cells with a polynucleotide encoding EGFRvIII has been correlated with a shift to a transformed cell morphology and with activation of phosphatidylinositol 3-kinase (PI 3-kinase) and the c-Jun N-terminal kinase (JNK) pathway. Antonyak et al. (1998), J. Biol. Chem. 273(5):2817-2822. Inhibition of PI 3-kinase or EGFR was correlated with a loss of a transformed cell morphology in cultured cells expressing EGFRvIII. Antonyak et al., supra; Moscatello et al. (1998), J. Biol. Chem. 273(1):200-206. Moscatello et al. (supra) suggest that the activation PI 3-kinase in EGFRvIII-expressing cells may play a role in conferring a selective advantage on EGFRvIII-expressing tumor cells in vivo. Antonyak et al. (1998), J. Biol. Chem. 273(5):2817-2822; Moscatello et al. (1998), J. Biol. Chem. 273(1):200-206.

Immunological methods are potentially useful in treating cancers that express either wild type EGFR or a rearranged version, such as EGFRvIII. For example, as explained above, antibodies against EGFR can serve as inhibitors of EGFR by inhibiting the binding of ligands to EGFR. Another immunological strategy involves the induction of immunity against antigens expressed on tumors by combining dendritic cells (DCs) ex vivo with one or more antigens expressed on tumor cells and then reintroducing these “pulsed” DCs into a living mammal affected with the tumor. See Heimberger et al. (2002), Neurosurgery 50 (1):158-64. Such antigens can be any protein expressed on a tumor cell or a peptide that is a portion of such a protein and is at least nine amino acids long. Preferably, the protein is expressed either preferentially or only on cancer cells. Such proteins or peptides are referred to herein as “immunogenic peptides.” An improvement of this method can include a step of “activating” the DCs by combining them ex vivo with a CD40 agonist (such as CD40 ligand (CD40L) or an antibody that binds to CD40), TNFα, lipopolysaccharides, granulocyte macrophage colony stimulating factor (GM-CSF), and/or monocyte conditioned medium (as described by Romani et al. (1996), J. Immunol. Methods 196(2):137-51 and Bender et al. (1996), J. Immunol. Methods 196(2):121-35). “Activation” will increase the number of MHC class I, MHC class II, and costimulatory molecules expressed on the surface of the DCs and increase the DCs' capacity to stimulate T cells. In addition, the number of DCs can be increased in vivo using Flt3-L and/or in vitro using GM-CSF plus IL-4 on blood monocytes or using Flt3-L with or without GM-CSF on hematopoietic progenitor cells from bone marrow or umbilical cord blood. These molecules and general approaches are described in U.S. Pat. Nos. 6,017,527, 5,554,512, 5,108,910, 5,229496, and U.S. Pat. No. 6,264,495, EP Patent No. 0 627 487, and WO 94/28391. Activated DCs are capable of activating CD8³⁰ T killer cells by presenting antigens to CD8³⁰ T killer cells. T killer cells recognize antigens via the T cell receptor proteins on their surface and are capable of killing cells displaying antigens in the MHC class I context recognized by their T cell receptors.

Tumors of the brain are difficult to treat by immunological methods because the brain is “immunologically privileged,” which means that many of the blood cells that mediate the immune response do not reach the brain. However, DCs that have been exposed to GM-CSF, mixed with a short peptide (PEP-3) that spans the deletion in the extracellular domain of EGFRvIII, and reintroduced into the bloodstream have been reported to elicit an immune response that dramatically improves the prognosis of mice whose brains have been injected with tumor cells expressing EGFRvIII. Generally, such DCs that have been exposed to an antigen ex vivo are referred to as “pulsed DCs” herein and can be prepared as described in Heimberger et al., supra, and Inaba et al. ((1992), J. Exp. Med. 176:1693-1702).

The instant invention provides a method for reducing tumor burden comprising the following steps: (a) administering to a human patient suffering from an EGFR⁺ cancer a therapeutically effective dose of an antibody that specifically binds to an antigen expressed on the cancer cells; (b) preparing DCs, preferably from the patient being treated, that is, autologous DCs; (c) combining the DCs with an antigen that is a peptide at least nine amino acids long and is substantially similar to part or all of EGFR or a tumor-specific variant thereof to yield “pulsed DCs;” and (d) introducing the “pulsed DCs” into the human patient suffering from an EGFR⁺ cancer.

In some embodiments, the antibody may specifically bind to EGFR or a tumor-specific variant thereof. If so, antibody binding can block EGFR ligand binding without activating EGFR, although an antibody that binds to a tumor-specific EGFR variant need not inactivate it if it is constitutively activated. This treatment may be particularly appropriate when the patient has a primary brain tumor or a brain metastasis. In some embodiments, the peptide used to “pulse” the DCs is substantially similar or identical to a portion of a tumor-specific version of EGFR that differs from the wild type EGFR. Alternatively, the peptide can be at least about 90% identical to a portion of a wild type EGFR. In either case, the peptide can be at least nine amino acids long so as to fit into the MHC class I binding site. The peptide can be longer than nine amino acids and can be cleaved to the appropriate size by MHC class I-associated proteases. The DCs can be (1) generated in vitro from peripheral blood monocytes by the addition of GM-CSF plus IL-4, (2) generated by administering Flt3-L to a mammal and subsequently recovering DCs from peripheral blood, or (3) grown from bone marrow or umbilical cord blood in the presence of Flt3-L and/or GM-CSF, with or without IL-4. The DCs may or may not be “activated” as described above.

The combined therapy provided by the invention can cause a number of functionally interrelated effects. The antibody can bind to tumor cells expressing the tumor antigen to which it specifically binds. In some embodiments, the antibody can bind specifically to EGFR or a tumor-specific variant thereof. Natural killer cells can kill cells that have antibodies bound to their surface. Flt3-L and GM-CSF can have pleitropic effects on DCs when used to stimulate the DCs ex vivo. First, they can stimulate the generation of DCs from peripheral blood monocytes or from stem cells such as those found in bone marrow or umbilical cord blood. Flt3-L and/or GM-CSF can also stimulate the production of Fc receptors on the surface of DCs, which stimulates phagocytosis of cells that have antibodies bound to their surface by DCs.

The phagocytosis of tumor cells by DCs can lead to the display of more tumor antigens and costimulatory molecules on the surface of DCs, which can ultimately lead to killing of tumor cells by T killer cells. Pulsed DCs that are “activated,” either ex vivo as described above or in vivo, can display the peptide with which they are pulsed in the context of MHC class I molecules, and activate T killer cells to kill other cells displaying the peptide in the MHC class I context. DCs in a “non-activated” state that display antigens in the MHC class I context and do not display costimulatory molecules are not very effective at eliciting an immune response. It is therefore advantageous to pre-activate the DCs ex vivo so that they will be in an activated state upon introduction into the host organism.

After phagocytosis of tumor cells, “activated” DCs displaying tumor peptides or proteins in the MHC class II context may also activate B cells to produce antibodies against the tumor-specific antigens. This can lead to increased killing of tumor cells by antibodies plus complement and/or antibodies plus natural killer cells, and/or by increased antibody-mediated phagocytosis by DCs, natural killer cells, granulocytes, and/or macrophages.

Phagocytosis of tumor cells by DCs can lead to the display of other tumor antigens by DCs, leading to further tumor cell killing by T killer cells that recognize these possibly unique tumor antigens in addition to EGFR or a tumor-specific variant thereof. Killing by complement constitutes another mechanism by which cells to which antibodies are bound are killed. See e.g. Frank and Fries, Complement, in Fundamental Immunology, Paul, ed., pp. 679-701, Raven Press, 1989. Phagocytosis can, in turn, lead to more display of tumor antigens in the MHC class I context, which can lead to more T killer cell killing of tumor cells, as described above. Thus, the multiple functions of DCs (including phagocytosis of antibody-coated cells, antigen display, possible activation of B cells producing tumor antigen-specific antibodies, and activation of tumor specific T killer cells) can produce a myriad of interrelated effects resulting from the proposed combination therapy.

Any antibody that specifically binds to an antigen expressed on EGFR⁺ tumor cells can be appropriate to practice this invention. As an example, therapeutically effective amounts of an antibody against CA125 (an antigen expressed in ovarian cancers), such as the murine antibody oregovomab (also called OVAREX® Mab (AltaRex Corp.)), plus DCs pulsed with part or all of EGFR or a tumor-specific variant thererof can be used to reduce tumor burden in patients with an EGFR⁺ ovarian cancer. As another example, therapeutically effective amounts of an antibody against HER2 (or c-erbB-2), such as the marketed antibody trastuzumab (trade name HERCEPTIN), plus DCs pulsed with part or all of EGFR or a tumor-specific variant thererof can be used to reduce tumor burden in a patient suffering from a breast cancer in which the cancer cells express both EGFR (c-erbB-1) and HER2 (c-erbB-2). Further, any of the antibodies described above that recognize EGFR or tumor-specific variants thereof are appropriate for practicing this invention.

Treatment and Prevention of Colorectal Cancer

Colorectal cancer (cancer of the large intestine and/or the rectum) is the second most common type of cancer and also the second most common cause of cancer death. Colon cancer usually begins with a button-like swelling on the surface of the intestinal lining or on a polyp. Polyps are growths with or without stalks that are usually noncancerous, but may be precancerous. The Merck Manual of Diagnosis and Therapy (1999), 17^{th} edition, Section 3. People with a hereditary condition known as familial adenomatous polyposis (FAP) are at a high risk of having their colorectal polyps transform from the adenoma to the carcinoma stage, that is, of contracting colorectal cancer. Almost all untreated people with FAP develop colon cancer before the age of forty. The Merck Manual of Diagnosis and Therapy, supra. Other conditions that increase risk of colorectal cancer include hereditary nonpolyposis colorectal cancer (HNPCC), the Lynch II syndrome, and hereditary flat adenoma syndrome (HFAS). Cohen et al., Colon Cancer, in Cancer: Principles & Practice of Oncology, pp. 929-977, 932-33, DeVita et al., eds., J.B. Lippincott Co., Philadelphia, (1993). Preventative treatment for high risk patients is desirable.

The invention provides a method for preventing or inhibiting the onset of colorectal cancer comprising administering a therapeutically effective amount of an EGFR inhibitor to a patient at high risk of developing colorectal cancer. Colorectal cancers can express EGFR, and increased EGFR expression is correlated with poor survival rates. Nicholson et al. (2001), Eur. J. Cancer 37 Suppl. 4:S9-15. EGF and TGFα can be autocrine and paracrine growth factors for gastric and colonic carcinomas. Moreover, transcription factors regulated by EGF and TGFa can promote transcription of gastrin, an effect that may or may not be mediated by EGFR. Watson et al. (2000), Int. J. Cancer 87:20-28. Moreover, gastrin phosphorylation, which may lead to cell proliferation in the gut, can be effected by a tyrosine kinase that is stimulated by EGF. Watson et al, supra. As explained below, gastrin can also be an autocrine and/or paracrine growth factor for gastric and colonic carcinomas. Thus, EGFR inhibitors can prevent or delay the onset of colorectal cancer through direct effects on cell proliferation and through effects on the transcription of the gastrin gene and the phosphorylation of gastrin.

Cancers ExPressing a Gastrin Receptor

Gastrin is a polypeptide molecular messenger that stimulates acid secretion and cell growth. It is important in normal and abnormal biological processes including maintenance of the gastric muscosa, proliferation of enterchromaffin-like cells, and neoplastic transformation. Gastrin can act as an autocrine and/or paracrine growth factor for gastrointestinal cancers. Smith et al. (2001), Proceedings of the American Society of Clinical Oncology 20: abstract #1029; Watson et al. (2000), Int. J. Cancer 87:20-28. It may play a similar role in other cancers that express the cholecystokininB/gastrin (CCK_{B}/gastrin) receptor. Schaer and Reubi (1999), J. Clin. Endocrinol. Metab. 84(1):233-39. Although the identity of the gastrin receptor that mediates the growth-promoting effects of gastrin in tumors is not certain, the CCK_{B}/gastrin receptor is a characterized gastrin receptor that may play this role. McWilliams et al. (2001), Regul. Pept. 99(2-3):157-61. The binding of either CCK or gastrin to their common receptor triggers the activation of multiple intracellular signaling pathways, including the extracellular regulated kinase (ERK) pathway, which promotes cell proliferation. Rozengurt and Walsh (2001), Annu. Rev. Physiol. 63:49-76. Such binding can also trigger a release of calcium from intracellular stores of calcium. Rozengurt and Walsh, supra at 56.

There are multiple regulatory and functional interconnections between gastrin and EGFR and its ligands. As mentioned above, transcription factors regulated by EGFR ligands can bind to the gastrin promoter and increase transcription of the gastrin gene. Watson et al. (2000), Int. J. Cancer 87:20-28. Also, gastrin can be phosphorylated by an EGF-stimulated tyrosine kinase, an event that has been hypothesized to lead to cell division, presumably mediated by an intracellular signaling cascade. Watson et al., supra. In addition, gastrin can promote the synthesis and processing of hb-EGF, the synthesis of amphiregulin RNA, and the tyrosine phosphorylation of EGFR in a rat gastric epithelial cell line. Since hb-EGF and amphiregulin are both EGFR ligands capable of activating EGFR, the observed phosphorylation is likely due to production of hb-EGF and/or amphiregulin. Miyazaki et al. (1999), Gastroenterology 116:78-89. Thus, although gastrin can activate multiple intracellular signaling pathways (Seufferlein et al. (1995), Cell Growth and Differentiation 6(4):383-93), at least some of its effects on growth may be due to increased production of EGFR ligands, leading to activation of EGFR. Moreover, effects of activation of EGFR on cell growth may be partially due to effects on gastrin phosphorylation in cells that express the gastrin receptor.

At least some effects of gastrin are mediated by its receptor, the CCK_{B}/gastrin receptor. Noble et al. (1999), Pharmacological Rev. 51(4):745-781, 752. CCK_{B}/gastrin receptors can be expressed in medullary thyroid carcinomas, small cell lung cancers, astrocytomas, stromal ovarian cancers, colorectal cancers, gastroenteropancreatic tumors, breast, endometrial, and ovarian adenocarcinomas and in normal brain, nervous system, and stomach tissue. Reubi et al. (1997), Cancer Res. 57(7):1377-86; Smith et al. (2000), Clinical Cancer Res. 6:4719-24; Noble et al. (1999), Pharmacological Rev. 51(4):745-781. Most cancers expressing CCK_{B}/gastrin receptors also produce gastrin. Reubi et al. supra. Elevation of circulating gastrin levels enhances the proliferation of normal colonic mucosa, resulting in an increase in the incidence of colorectal cancer in patients with elevated levels of gastrin. Smith et al., supra. Antagonists of the CCK_{B}/gastrin receptor may block these effects of elevated levels of gastrin and may also have other effects, such as a lessening of anxiety, depression, and/or perception of pain, effects that are presumably due to the widespread expression of CCK_{B}/gastrin receptors in the central nervous system. Noble et al., supra.

The invention provides a method for reducing tumor burden comprising administering a therapeutically effective amount of an EGFR inhibitor to a human patient suffering from a cancer in which the cancer cells express the CCK_{B}/gastrin receptor and do not overexpress EGFR or, alternatively, express little or no EGFR. In EGFR⁺ cancers that do not overexpress EGFR, EGFR may be responsible for mediating at least some of the positive effects of gastrin on growth. Thus, EGFR inhibitors can be particularly useful for treating tumors expressing gastrin receptors. EGFR inhibitors can inhibit positive effects on growth due to the binding of EGFR ligands, some of which are upregulated when cells expressing CCK_{B}/gastrin receptors are exposed to gastrin. Also, EGFR inhibitors may reduce the phosphorylation of the gastrin protein.

In addition, the invention provides a method for reducing tumor burden comprising administering a therapeutic amount of a small molecule EGFR inhibitor to a patient suffering from a cancer in which the cancer cells express the CCK_{B}/gastrin receptor, but not EGFR. Such a treatment may work through non-specific effects of the small molecule EGFR inhibitor on tyrosine kinases that are part of one or more intracellular signaling cascades. Other mechanisms of action are also possible.

The invention further provides a method for reducing tumor burden comprising administering therapeutically effective amounts of an EGFR inhibitor and an inhibitor of the interaction between gastrin and the CCK_{B}/gastrin receptor to a patient suffering from an EGFR⁺ cancer, in which the cancer cells express the CCK_{B}/gastrin receptor and may or may not overexpress EGFR. Such a combination can inhibit EGFR directly, reduce production of at least one EGFR ligand, and/or inhibit the effects of gastrin binding to the CCK_{B}/gastrin receptor. In addition, beneficial central nervous system effects of inhibitors of the CCK_{B}/gastrin receptor, such as alleviation of anxiety, depression, and/or perception of pain, can also benefit the patient.

Inhibitors of the interaction between gastrin and the CCK_{B}/gastrin receptor include: antibodies that bind to either gastrin or the CCK_{B}/gastrin receptor, thereby inhibiting this receptor/ligand interaction (see McWilliams et al. supra) without activating the CCK_{B}/gastrin receptor; polypeptides comprising part or all of the CCK_{B}/gastrin receptor that can bind to gastrin; small molecules that inhibit the binding of gastrin to CCK_{B}/gastrin receptor; inhibitors of the interactions between CCK_{B}/gastrin receptor with downstream effector molecules that lead to the activation of, for example, the ERK pathway; inhibitors of the interactions between CCK_{B}/gastrin receptor with downstream effector molecules that lead to the tyrosine phosphorylation of focal adhesion kinase (p125 fak), cell adhesion kinase β (Pyk-2), related adhesion focal tyrosine kinase, calcium-dependent protein tyrosine kinase, Src, Crk-associated substrate(CAS), and/or paxillin; proteins, nucleic acids (such as antisense molecules, ribozymes, DNA enzymes, triple helix-forming nucleic acids, or interfering RNAs), or small molecules that interfere with the expression of gastrin or the CCK_{B}/gastrin receptor; vaccines that elicit an immune response to either gastrin or the CCK_{B}/gastrin receptor; and inhibitors of the interactions between CCK_{B}/gastrin receptor with downstream effector molecules that lead to the failure of gastrin binding to elicit a release of calcium from intracellular stores of calcium. Examples of such inhibitors include: the vaccine G17DT (Watson and Gilliam (2001), Expert Opin. Biol. Ther. 1(2):309-17), which produces antibodies directed against gastrin; the “peptoid” analogue of CCK, CI-1015 (Trivedi et al. (1998), J. Med. Chem. 4(1):38-45); tetronothiodin and virginiamycin analogues isolated from Streptomyces (Jensen (1996), Yale J. Biol. Med. 69(3):245-59); ureido-acetamide analogues RP 69758, RP 72540, and RP 73870 (Jensen, supra; Ding and Hakanson (1996), Pharmacol. Toxicol. 79(3): 124-30); the benzodiazepine analogues L-740,093, L-365,260, L-368,935, YM022 (Jensen, supra; Ding and Hakanson, supra); pyrazolidimine analogues including LY 262,691; and glutamic acid analogues including CR2194, among others. Watson and Gilliam (2001), Expert Opin. Biol. Ther. 1(2):309-17; McWilliams et al. (2001), Regul. Pept. 99(2-3):157-61; Noble et al. supra. Appropriate doses for gastrin inhibitors can be determined by one of skill in the art. As an example, appropriate doses for G17DT range from 5 mg to 700 mg, optionally from 50 mg to 500 mg, or from 100 to 250 mg and are repeated more than once. One of skill in the art will realize that when EGFR inhibitors and gastrin inhibitors are used as a combination, doses for either or both may be lower or higher than doses that are appropriate for each when used as a single agent.

Ovarian Cancer

EGFR expression likely results in multiple phenotypic changes that could enhance the invasive phenotype. Alper et al. (2001), J. Natl. Cancer Inst. 93(18):1375-84; Brady et al. (1998), Oncol. Rep. 5(5):1269-74. Moreover, suppression of EGFR expression in ovarian carcinoma cells with antisense RNA results in decreased tumorogenicity of these cells in nude mice. Alper et al. (2000), Int. J. Cancer 88(4):566-74. Accordingly, the invention provides a method for reducing tumor burden comprising administering therapeutically effective amounts of an EGFR inhibitor and an antibody that specifically binds to an antigen that is preferentially expressed on ovarian cancer cells to a human patient suffering from an EGFR⁺ ovarian cancer.

In one embodiment, invention provides a method for reducing tumor burden comprising administering therapeutically effective amount of an EGFR inhibitor and an antibody that specifically binds to CA125 to a human patient suffering from an EGFR⁺ ovarian cancer in which the cancer cells express CA125. CA125 is a 200 kilodalton glycoprotein found on the surface of most ovarian cancer cells. See e.g. Cancer: Principles and Practice of Oncology, 4^{th} Edition, DeVita et al., eds., J.B. Lippincott Co., Philadelphia, Pa., pp. 534-35 (1993); Davis et al. (1986), Cancer Res. 46(12pt1):6143-48; Yin and Lloyd (2001), J. Biol. Chem. 276(29):27371-75. Most ovarian carcinomas express high levels of EGFR.

As an alternative, the invention provides a method for reducing tumor burden comprising administering therapeutically effective doses of an antibody against HPP14 and an EGFR inhibitor to a human patient suffering from an EGFR⁺ ovarian cancer in which the cancer cells express both HPP14. HPP14 is an ovarian cancer antigen described in US Patent Application No. 2002/0064815.

Suitable antibodies against CA125 include the murine antibody oregovomab (also called OVAREX® Mab (AltaRex Corp.)), the murine monoclonal antibody NCL-CA125 (sold by Novocastra Laboratories Ltd., Newcastle upon Tyne, UK), one of the many murine anti-CA125 antibodies sold by Biodesign International (Saco, Me., USA), such as M37201M, M37197, M37930M, etc., and a humanized or fully human antibody that binds specifically to CA125. When administered to a human, the murine anti-CA125 antibody OV can induce an immune response against both the antibody itself and its target CA125. The existence of a substantial immune response of this kind correlates with a prolonged time to relapse in human patients. Berek et al. (2001), Am. Soc. Clin. Oncol. Programs and Proceedings, Abstract # 837.

Prostate Cancer

Prostate cancer cells may or may not express EGFR or not and may be androgen dependent, sensitive, or independent. Initially, prostate cancer cells are usually androgen dependent, but, if the disease progresses, they usually become androgen independent, an event that is usually lethal. Feldman and Feldman (2001), Nature Reviews Cancer 1:3445. Treatment of androgen independent cancers with androgen ablation is, of course, ineffective. Decreases in the levels of epidermal growth factor receptor expression and in reactivity to testosterone have been shown to correlate with more aggressive, rapidly growing tumors. Rubenstein et al. (1991), Urol. Res. 19:309-12. GM-CSF can stimulate the expression of epidermal growth factor receptor and increase testosterone reactivity in androgen independent prostate cancer cells in vivo, characteristics typical of slower-growing, hormone-sensitive prostate cancer cells. Rubenstein et al. (1991), Urol. Res. 19(5):309-12.

The invention provides a method for reducing tumor burden comprising administering therapeutically effective amounts of an EGFR inhibitor and GM-CSF to a patient suffering from prostate cancer. The prostate cancer cells may or may not express EGFR and may be androgen dependent, sensitive, or independent. GM-CSF can be administered at doses from about 25 mg to about 700 mg, optionally from about 50 mg to about 500 mg, or from about 100 mg to about 400 mg, or from about 200 mg to about 300 mg at intervals from daily to monthly. One of skill in the art will realize that when EGFR inhibitors and GM-CSF are used as a combination, doses for either or both may be lower or higher than doses that are appropriate for each when used as a single agent.

Cerebral Ischemia

Cerebral ischemia is the impairment of blood flow to the cerebrum and can be commonly caused by atherosclerosis, hypertension, arteritis, rheumatic heart disease, and/or congenital abnormalities and less commonly caused by the use of sympathomimetic drugs such as cocaine or amphetamines or the compression of a blood vessel by a bony projection. Atheroschlerosis is characterized by patchy thickening of medium and large arteries, which can obstruct blood flow. Hypertension may cause impairment of blood flow (or ischemia) due to spasm of blood vessels caused by increases in blood pressure. Dickinson (2001), J. Hypertension 19:1515-21. Sudden insufficiency of blood supply due to ischemia or other causes can cause an area of necrosis, known as an infarct, in the wall of a blood vessel. Cerebral ischemia can result in transient ischemic attacks or stroke. Merck Manual of Diagnosis and Therapy (1999), 17^{th} ed., section 14. A transient ischemic attack typically lasts from about 2 to 30 minutes and is usually without persistant after-effects, although infarcts do result in some cases. Symptoms of an ischemic attack are similar to those of a stroke and may include confusion, vertigo, blindness, double vision, weakness, slurred speech, and impaired ability to comprehend and communicate. Patients who have experienced a transient ischemic attack have an increased risk for stroke. A stroke is the sudden development of localized defects in the brain, which may last from several hours to a day or two, and may be caused by an enlarging brain infarct (ischemic stroke) or an intracerebral hemorrhage resulting from rupture of a blood vessel. Strokes can be fatal, but symptoms (mentioned above) often abate soon after, with further improvement in the following days to months. Available evidence indicates that hemorrhage arises from previous infarcts. Dickinson (2001), J. Hypertens. 19(9):1515-21. Cerebral ischemia likely promotes the occurrence of either of these kinds of strokes.

The occurrence of hypertension correlates with increased risk of stroke (Dickinson, supra). It has been reported that both EGFR and EGF messenger RNAs are more highly expressed in cerebral and other blood vessels of hypertensive rats than they are in normal rats. Sambhi et al. (1992), Biochem. Med. Metab. Biol. 48(1):8-18; Dorrance et al. (2001), Amer. J. Physiol.—Regulatory Integrative & Comparative Physiol. 281(3):R944-50. Furthermore, binding of EGF by aortic membranes is increased in hypertensive rats when compared to normal rats. Sambhi et al., supra; Swaminathan et al. (1996), Clin. Exp. Pharmacol. Physiol. 23:793-96. Also, cultured T lymphocytes isolated from human peripheral blood up-regulate mRNA encoding another EGFR ligand, hb-EGF, in response to lysophosphatidylcholine (a polar phospholipid component present in atherosclerotic lesions), suggesting that such growth factors may modulate atherosclerotic progression. Nishi et al. (1997), Circulation Res. 80(5):638-44. Thus, EGFR or its ligands may play roles in maintaining hypertension and in the progression of atherosclerosis, both of which can cause cerebral ischemia. Accordingly, the invention provides a method to prevent or to reduce the frequency and/or severity of transient ischemic attacks and/or strokes comprising administering to a patient suffering from cerebral ischemia a therapeutically effective amount of an EGFR inhibitor.

Hematologic Cancers

The invention provides a method for reducing tumor burden comprising administering to a patient suffering from a hematologic cancer therapeutically effective amounts of an EGFR inhibitor and of another anti-neoplastic agent. The hematologic cancer cells may express little or no EGFR. The anti-neoplastic agent may be a chemotherapeutic agent or a non-chemotherapeutic anti-neoplastic agent. The anti-neoplastic agent may not be a farnesyl transferase inhibitor.

The invention further provides a method for reducing tumor burden comprising administering to a patient suffering from a hematologic cancer therapeutically effective amounts of an EGFR inhibitor and of an antibody that can bind to an antigen that is expressed on the hematologic cancer cells.

Further, the invention provides a method for reducing tumor burden comprising administering to a patient suffering from a hematologic cancer therapeutically effective amounts of an EGFR inhibitor and a chemotherapeutic agent. In this embodiment, the hematologic cancer cells express little or no EGFR.

EGFR is normally expressed on at least two types of cells that can be found in bone marrow, bone marrow stromal cells and erythrocytic progenitor cells. In both cell types, EGFR likely plays a role in promoting cell proliferation and inhibiting differentiation. Satamura et al. (1998), J. Cell. Physiol. 177:426-38; Pain et al. (1991), Cell 65(1):37-46. Although EGFR is not usually expressed by hematologic tumors, EGFR ligands are produced by some hematologic tumors. See e.g. Wu et al. (1995), Internatl. J. Hematol. 62(2):83-9; Walz et al. (1993), Cancer Res. 53(1): 191-96. Expression of these ligands by cancer cells can influence the growth and differentiation of cells in the bone marrow that do express EGFR, upsetting the normal homeostasis of cell proliferation versus cell differentiation within the bone marrow. Inhibiting EGFR may shift the homeostasis within the bone marrow, making hematologic tumors within the bone marrow more susceptible to therapy by antibodies targeted to antigens expressed on hematologic tumor cells or other anti-neoplastic agents.

Hematologic neoplasias and neoplastic-like conditions include all diseases characterized by abnormal proliferation of cells found in blood or hemopoietic tissues. These include, but are not limited to: Hodgkins lymphoma; non-Hodgkins lymphomas (including Burkitt's lymphoma, small lymphocytic lymphoma, chronic lymphocytic leukemia, mycosis fungoides, mantle cell lymphoma, follicular lymphoma, diffuse large B-cell lymphoma, marginal zone lymphoma, hairy cell leukemia, and lymphoplasmacytic leukemia); tumors of lymphocyte precursor cells (including B-cell acute lymphoblastic leukemia/lymphoma and T-cell acute lymphoblastic leukemia/lymphoma); thymoma, tumors of the mature T and NK cells (including peripheral T-cell leukemias, adult T-cell leukemia/T-cell lymphomas and large granular lymphocytic leukemia); Langerhans cell histocytosis, myeloid neoplasias such as acute myelogenous leukemias (including AML with maturation, AML without differentiation, acute promyelocytic leukemia, acute myelomonocytic leukemia, and acute monocytic leukemias); acute lymphoblastic leukemias; eosinophilic leukemias; megakaryocytic leukemias; meningeal leukemias; micromyeloblastic leukemias; neutrophilic leukemias; plasma cell leukemias; polymorphocytic leukemias; splenic leukemias; myelodysplastic syndromes; and chronic myeloproliferative disorders (including chronic myelocytic leukemias).

Suitable antibodies against antigens expressed on hematologic tumor cells include single- or multi-chain antibodies or fragments thereof. In particular embodiments, the antibody may be conjugated to a luminescent, radioactive, and/or cytotoxic substance, for example, maytansine derivatives (such as DM1), enterotoxins (such as a Staphlyococcal enterotoxin), iodine isotopes (such as iodine-125), technetium isotopes (such as Tc-99m), cyanine fluorochromes (such as Cy5.5.18), or ribosome-inactivating proteins (such as bouganin, gelonin, or saporin-S6). Examples of suitable antibodies include: an anti-CD33 antibody conjugated to calicheamicin, a cytotoxic substance, marketed under the trademark MYLOTARG™ (Wyeth-Ayerst); ¹³¹I-labeled anti-CD45 antibodies; an anti-CD52 antibody (CAMPATH-1H®) alone or in combination with an anti-CD20 antibody (RITUXIMAB®). Suitable doses of an anti-CD33 antibody conjugated to calicheamicin (MYLOTARG™) range from 0.1 mg/m² to 100 mg/m², optionally from 1 mg/m² to 20 mg/m², or about 9 mg/m² administered at, for example, 14 day intervals. See e.g. Sievers et al. (2001), American Society of Clinical Oncology, Programs/Proceedings, Abstract #1204; Stadtmauer et al. (2001), American Society of Clinical Oncology, Programs/Proceedings, Abstract #1203. Suitable doses for an anti-CD52 antibody (CAMPATH-1H®) range from 1 mg to 100 mg, optionally from 3 mg to 30 mg administered, for example, daily, every other day, three times per week, twice per week, once per week, or every other week, among other possible dosage schedules. See e.g. Keating et al. (2002), J. Clin. Oncol. 20(1):205-13. An anti-CD20 antibody (RITUXIMAB®) can be administered in doses ranging from 150 mg/m² to 600 mg/m², optionally from 300 mg/m² to 450 mg/m², or about 375 mg/m² administered, for example, weekly, among other possible dosage schedules. See e.g. Igarishi et al. (2001), American Society of Clinical Oncology, Programs/Proceedings, Abstract #2154; Gupta et al. (2001), American Society of Clinical Oncology, Programs/Proceedings, Abstract #1133.

Benign Prostatic Hyperplasia

Benign prostatic hyperplasia involves inappropriate growth of cells in the region of the prostate gland that can obstruct urine outflow, which may ultimately compromise renal function. Human prostate stromal cells can express both EGFR and one of its ligands, hb-EGF, both of which may play a role in proliferation of stromal cells in benign prostatic hyperplasia. Duque et al. (2001), J. Urol. 165(1):284-88. Thus, EGFR inhibitors can inhibit proliferation of prostate cells that express EGFR, thereby ameliorating the symptoms of benign prostatic hyperplasia. The invention encompasses a method for reducing the size of an enlarged prostate comprising administering a therapeutically effective amount of an EGFR inhibitor to a patient suffering from benign prostatic hyperplasia.

Endometriosis

Endometriosis is a non-malignant disorder in which cells that appear to be endometrial cells (that is, from the interior lining of the uterus) are present outside the uterus, that is, at ectopic sites. Commonly, this ectopic growth occurs on the surfaces of abdominal organs such as the ovaries, the posterior broad ligament, the posterior cul-de-sac, and uterosacral ligaments. Symptoms of endometriosis include pelvic pain, pelvic mass, alteration of menses, and infertility. Some data indicate that endometrial cells express EGFR and that a decrease in stage of progression of endometriosis correlates with a decrease in EGFR expression. Huang and Yeh (1994), J. Clinical Endocrinol. Metab. 79(4):1097-101; Di Lieto et al. (1997), Gynecol. Endocrinol. 11(1):17-20. The invention provides a method for (1) reducing the size of one or more masses of endometrial cells at one or more ectopic sites, (2) reducing pain, (3) reducing menstrual abnormalities, and/or (4) correcting infertility. The method comprises administering a therapeutically effective amount of an EGFR inhibitor to a patient suffering from endometriosis.

Gross Cystic Disease

Gross cystic disease is a benign condition characterized by the presence of one or more fluid-filled cysts in the breast greater than 3 millimeters in diameter. Although it is not a cancer, women who have had gross cystic disease are at increased risk of developing breast cancer, particularly if the level of EGF in the cyst fluid is high. Boccardo et al. (2001), Int. J. Cancer (Pred. Oncol.) 95:260-65; Hess et al. (1994), Amer, J. Surgery 167:523-30. In addition, breast cyst fluid has been shown to contain growth factors including EGF, insulin-like growth factors I and II, platelet-derived growth factor, and transforming growth factor β. Hess et al., supra. Moreover, clinical improvement in patients affected by Benign Breast Disease (a designation that includes gross cystic disease) correlates with a reduction in EGFR expression. Di Lieto et al. (1996), Clin. Exp. Obstet. Gynecol. 23(4):220-28. Accordingly, a method is further provided for lessening symptoms of gross cystic disease of the breast, inhibiting recurrence, and/or inhibiting the progression from gross cystic disease of the breast to breast cancer comprising administering to a human patient who presently has or has had gross cystic disease of the breast a therapeutically effective amount of an EGFR inhibitor. Small molecule EGFR inhibitors can be particularly appropriate for treating this condition because they can, in most cases, be administered orally.

Polycvstic Kidney Disease

Polycystic kidney disease is characterized by cysts in the kidneys that can lead to grossly enlarged kidneys in advanced cases. Initially, it is often asymptomatic. When symptoms do occur, they include lumbar discomfort or pain, blood in the urine, urinary tract infection, and abdominal pain. Calculi, which are solid particles or “stones,” are often present in the kidneys of patients affected with polycystic kidney disease. Polycystic kidney disease may be autosomal dominant or autosomal recessive, and a mutation present in most autosomal dominant families has been localized to chromosome 16. The The Merck Manual of Diagnosis and Therapy (1999), 17^{th} edition, Section 230. In addition, evidence from a variety of sources suggests that EGFR and its ligands play a role in polycystic kidney disease. See e.g. Lakshmanan and Eysselein (1993), Biochem. Biophys. Res. Commun. 197:1083-93; Sweeney and Avner (1998), Am. J. Physiol. 275(3)pt.2:F387-94; Klingel et al. (1992), Am. J. Kidney Dis. 19:22-30; Wilson (1991), Am. J. Kidney Dis. 17:634-47; Horikoshi et al. (1991), Kidney Int. 39(1):57-62.

The instant invention provides a method for (1) reducing the size or number of cysts, (2) reducing lumbar discomfort, (3) reducing the amount of blood in the urine or the frequency with which blood is found in the urine, (4) reducing the size or number of calculi in the kidneys, (5) reducing the frequency or severity of urinary tract infections, and/or (6) reducing the severity of symptoms or the progression of disease in a patient suffering from polycystic kidney disease. The method comprises administering a therapeutically effective amount of an EGFR inhibitor to a patient suffering from polycytic kidney disease.

Tumors that Express CD40

CD40 is expressed on some hematopoietic cells and on some carcinoma cells, including ovarian carcinoma cells. Gallagher et al. (2002), Mol. Pathol. 55(2): 110-20. Ligation of CD40 by adding to ovarian carcinoma cells that express CD40 a soluble version of CD40 ligand (CD40L) leads to growth inhibition of these cells. In addition, apoptosis occurs when both CD40 ligand and a protein synthesis inhibitor (cycloheximide) are added. Gallagher et al. supra. Burkitt's lymphoma cells can also be growth arrested by CD40 activation. However, in normal B cells, activation of the CD40 pathway provides an anti-apoptotic and proliferative stimulus. Thus, responses to CD40 agonists appear to be cell-specific.

Accordingly, the invention provides a method for reducing tumor burden comprising administering to a patient suffering from an EGFR⁺ cancer therapeutically effective amounts of a CD40 agonist and an EGFR inhibitor. The cancer can, for example, be a hematologic cancer or an ovarian cancer, and the cancer cells can express CD40. CD40 agonists include, but are not limited to, the following molecules: polypeptides comprising all or part of the CD40L or substantially similar polypeptides that can serve as agonists of CD40; part or all of an antibody or a substantially similar polypeptide that can specifically bind to CD40 and serve as an agonist of CD40, such as those described in U.S. Pat. Nos. 5,801,227, 5,677,165, or U.S. Pat. No. 5,874,082, among others; part or all of a polypeptide selected for CD40 binding in vitro or a substantially similar polypeptide that can serve as an agonist to CD40; CD40 antibodies, agonists, and binding proteins described in U.S. Pat. No. 5,801,227 and U.S. Pat. No. 5,674,492; and small molecules that can serve as CD40 agonists. In vitro selection schemes to obtain binding proteins are described in e.g. He and Taussig ((1997), Nucleic Acids. Res. 25(24):5132-5134), Hanes and Pluckthun ((1997), Proc. Natl. Acad. Sci. 94:4937-4942), Roberts and Szostak ((1997), Proc. Natl. Acad. Sci. 94:12297-12302), Lohse and Wright ((2001), Curr. Opin. Drug Discov. Devel. 4(2):198-204), Kurz et al. ((2000), Nucleic Acids Res. 28(18):E83), Liu et al. ((2000), Methods Enzymol. 318:268-93), Nemoto et al. ((1997), FEBS Lett. 414(2):405-08), U.S. Pat. No. 6,261,804, WO 00/32823, WO 00/34784, Parmley and Smith ((1989), Adv. Exp. Med. Biol. 251:215-218), Luzzago et al. ((1995), Biotechnol. Annu. Rev. 1:149-83), and Lu et al. ((1995), Biotechnology (NY) 13(4):366-372). The sequences of CD40 and CD40L are known in the art. See Stamenkovic et al. (1989), EMBO J. 8:1403-10; Spriggs et al. (1992), J. Exp. Med. 176:1543-50. Methods for making antibodies are also known in the art. A variety of standard assays have been described for assessing whether a particular molecule can agonize CD40. Several assays for apoptosis, which CD40L prevents in osteoclasts, are described in WO 01/16180 and in Gallagher et al., supra. Assays for cell proliferation, such as cell counting and optical density measurements, are well known in the art. Assays for expression of specific genes (as described in Gallagher et al. supra) can also be indicative of agonism of CD40.

Therapeutic Proteins

Encompassed by the present invention are proteins comprising part or all of at least one of the therapeutic proteins described above or substantially similar proteins that are oligomers and/or that are fused to non-polypeptide molecules that enhance in vivo half life or activity, such as polyethylene glycol or various sugar or carbohydrate moieties, among others. Oligomers may be in the form of covalently-linked or non-covalently-linked dimers, trimers, or higher order oligomers. One embodiment of the invention is directed to oligomers comprising multiple proteins, at least one of which can inhibit EGFR, joined via covalent or non-covalent interactions between peptide moieties. Such interactions may be effected by a peptide linker (spacer) or by stretches of amino acids that have the property of promoting oligomerization. For example, leucine zippers and certain polypeptides derived from antibodies can promote oligomerization of proteins attached thereto.

In particular embodiments, the oligomers can comprise from two to four of the proteins described above that can inhibit EGFR. Examples of such proteins include the following proteins or substantially similar proteins: antibodies or non-antibody proteins that can bind to EGFR or at least one of its ligands; soluble forms of EGFR that can bind at least one EGFR ligand (such as part or all of the extracellular region of EGFR); and proteins useful for generating an immune response against cells expressing EGFR (such as those described in U.S. Pat. No. 5,894,018 and U.S. Pat. No. 6,224,868). The EGFR-inhibiting moieties of the oligomer can be soluble proteins.

Alternatively, a particular embodiment can comprise all or part of the following oligomeric proteins or substantially similar proteins: the proteins capable of inhibiting TNF described above; KGF-1 or −2; the RANKL inhibitors described above; an antibody against a antigen that is preferentially expressed on tumor cells as described above in connection with pulsed dendritic cells, such as oregovomab or trastuzumab; proteins that inhibit the interaction between gastrin and the CCK_{B}/gastrin receptor as described above; the anti-CA125 antibodies described above; anti-CD33 antibodies; anti-CD45 antibodies; anti-CD52 antibodies; anti-CD40 antibodies; and/or anti-CD20 antibodies, among others.

In some embodiments, an oligomer can be prepared using polypeptides derived from immunoglobulins fused to non-antibody proteins. Preparation of fusion proteins comprising certain heterologous proteins fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo ((1992), “Construction of Immunoglobulin Fusion Proteins”, in Current Protocols in Immunology, Suppl. 4, pages 10.19.1-10.19.11).

One embodiment of the present invention is directed to a human IgG antibody against EGFR, ABX-EGF, which is tetrameric antibody against EGFR consisting of two antibody heavy chains and two light chains produced by the hybridoma cell line E7.6.3, as described in U.S. Pat. No. 6,235,883B1 and WO 98/50433. Another embodiment is a dimer comprising two fusion proteins created by fusing a soluble portion of any of the above-mentioned proteins or a substantially similar protein, for example, all or part of the extracellular region of EGFR, to an Fc region of an antibody or a substantially similar protein. A gene fusion encoding such a fusion protein can be inserted into an appropriate expression vector. Fusion proteins are expressed in host cells transformed with the recombinant expression vector, and allowed to assemble much like antibody molecules, whereupon interchain disulfide bonds form between the Fc moieties to yield dimers. Truncated forms of such polypeptides containing the hinge region that promotes dimerization are also included. Moreover, heterodimers comprising all or part two different proteins, for example, both EGFR and c-erbB-2 (HER2), are also encompassed by the invention. Such heterodimers can be useful as inhibitors of EGFR. The above-described fusion proteins comprising Fc moieties (and oligomers formed therefrom) offer the advantage of facile purification by affinity chromatography over Protein A or Protein G columns. In other embodiments, all or part of one of the non-antibody proteins described above may be substituted for the variable portion of an antibody heavy and/or light chain.

Alternatively, the oligomer can be a fusion protein comprising all or part of more than one of the above-mentioned proteins, with or without peptide linkers (spacer peptides). Among the suitable peptide linkers are those described in U.S. Pat. No. 4,751,180 and U.S. Pat. No. 4,935,233, which are hereby incorporated by reference. A DNA sequence encoding a desired peptide linker may be inserted between, and in the same reading frame as, the DNA sequences encoding all or part of the desired proteins, using any suitable conventional technique. For example, a chemically synthesized oligonucleotide encoding the linker may be ligated between sequences encoding the extracellular portions of EGFR and c-erbB-2 (HER2).

Another method for preparing oligomers involves use of a leucine zipper. Leucine zipper domains are peptides that promote oligomerization of the proteins in which they are found. Leucine zippers were originally identified in several DNA-binding proteins ((1988), Landschulz et al., Science 240:1759), and have since been found in a variety of different proteins. Among the known leucine zippers are naturally occurring peptides and derivatives thereof that dimerize or trimerize.

Examples of leucine zipper domains suitable for producing soluble oligomeric proteins are described in WO 94/10308, and the leucine zipper derived from lung surfactant protein D (SPD) described in Hoppe et al. ((1994), FEBS Letters 344:191). The use of a modified leucine zipper that allows for stable trimerization of a heterologous protein fused thereto is described in Fanslow et al. ((1994), Semin. Immunol. 6:267-278). Recombinant fusion proteins comprising a soluble polypeptide fused to a leucine zipper peptide are expressed in suitable host cells, and the soluble oligomeric protein that forms is recovered from the culture supernatant.

Therapeutic Methods and Administration

Treatment of a cell proliferative disease, including cancer, encompasses alleviation of at least one symptom of the disorder, a reduction in the severity of the disease, or the delay or prevention of progression to a more serious disease that occurs with some frequency following the treated condition. Treatment need not mean that the disease is totally cured. A useful therapeutic agent needs only to reduce the severity of a disease, reduce the severity of symptom(s) associated with the disease or its treatment, or delay the onset of a more serious disease that can occur with some frequency following the treated condition. For example, if the disease is a cancer, a therapeutic agent may reduce the tumor burden, that is, reduce the number of viable cancer cells, the number of tumor sites, and/or the size of one or more tumors. A patient's tumor burden may be assessed by any of a number of conventional techniques. Suitable procedures vary according to the type of cancer, but include various tumor imaging techniques, or procedures for determining the amount of a given tumor-associated antigen or protein in a patient's blood or urine, for example. The invention encompasses a method of treatment comprising administering to a patient afflicted with a cell proliferative disease an EGFR inhibitor in an amount and for a time sufficient to induce a sustained improvement over baseline of an indicator that reflects the severity of a particular disorder or the severity of symptoms caused by the disorder or to delay or prevent the onset of a more serious disease that follows the treated condition in some or all cases. In some embodiments, the invention further comprises the administration of another therapeutic agent in an amount and for a time sufficient to induce a sustained improvement over baseline of an indicator that reflects the severity of the disorder or the severity of symptoms caused by the disorder or its treatment or to delay or prevent the recurrence or the onset of a more serious disease that follows the treated condition in some or all cases.

Any of the above-described cancer treatments can be administered with a variety of drugs and treatments have been widely employed in cancer treatment such, for example, chemotherapy and/or radiation. For example, chemotherapy and/or radiation can occur before, during, and/or after any of the treatments described herein. Examples of chemotherapeutic agents include, but are not limited to, cisplatin, taxol, etoposide, mitoxantrone (Novantrone®, Amgen Corporation, Thousand Oaks, Calif.), actinomycin D, cycloheximide, camptothecin (or water soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned above.

Derivatives of camptothecin that are more water soluble are advantageous for in vivo use. Examples of such water soluble derivatives are the drugs 7-ethyl-10-[4-(1-piperidino)-1-piperidino]carbonyloxy-camptothecin (CPT-11; irinotecan) and 9-dimethyl-aminomethyl-10-hydroxycamptothecin (topotecan). Camptothecin and the two above-described derivatives are DNA topoisomerase I inhibitors.

Drugs employed in cancer therapy may have a cytotoxic or cytostatic effect on cancer cells, or may reduce proliferation of the malignant cells. Cancer treatment may include radiation therapy. In particular embodiments, any of the above-described treatments may be co-administered with other non-chemotherapeutic anti-neoplastic agents used in cancer therapy; one such agent is γ-interferon.

Among the texts providing guidance for cancer therapy is Cancer, Principles and Practice of Oncology, 4^{th} Edition, DeVita et al., Eds. J. B. Lippincott Co., Philadelphia, Pa. (1993). An appropriate therapeutic approach is chosen according to the particular type of cancer, and other factors such as the general condition of the patient, as is recognized in the pertinent field. The treatments described herein may be added to a therapy regimen using other anti-neoplastic agents in treating a cancer patient.

Any of the above-described therapeutic agents can be administered in the form of a composition, that is, with one or more additional components such as a physiologically acceptable carrier, excipient, or diluent. For example, a composition may comprise an EGFR inhibitor as described herein plus a buffer, an antioxidant such as ascorbic acid, a low molecular weight polypeptide (such as those having less than 10 amino acids), a protein, amino acids, carbohydrates such as glucose, sucrose, or dextrins, chelating agent such as EDTA, glutathione, and/or other stabilizers, excipients, and/or preservatives. The composition may be formulated as a liquid or a lyophilizate. Further examples of components that may be employed in pharmaceutical formulations are presented in Remington's Pharmaceutical Sciences, 16^{th} Ed., Mack Publishing Company, Easton, Pa., (1980).

In some embodiments, any of the above-described therapeutic agents may be delivered to patients in the form of a kit containing everything necessary to administer the chosen therapeutic agents, including the therapeutic agent(s) to be administered and any apparatus necessary for administration. For example, if at least one of the agents to be administered is to be injected, the kit can contain an injection apparatus, one or more therapeutic agents to be administered, and a suitable diluent if a therapeutic agent to be injected is in a dry form. Alternatively, an injectable therapeutic agent may be supplied in liquid form. Furthermore, if more than one therepeutic agent is to be administered, more than one therapeutic agent can be present in a composition to be administered. The therapeutic agents present in such a composition can be effective when administered by the same route.

Any of the above-described therapeutic agents or combinations of agents can be commercially supplied as a kit containing an EGFR inhibitor as described above in a form suitable for administration (possibly as part of a composition), any devices and/or supplies necessary for administration, and, for some of the above-described inventions, a second therapeutic agent in a form suitable for administration (possibly as part of a composition). The EGFR inhibitor and the second agent may be mixed in a single composition. Devices and/or supplies included in such a kit can include: syringes and needles for injection; special injection devices designed to both prepare a composition for injection, i.e. by dissolving it in a suitable diluent, and inject the composition such as, for example, those described in U.S. Pat. No. 6,364,865 and U.S. Pat. No. 6,319,225, among others; skin patches for admininistering therapeutic agents transdermally, accompanied by suitable adhesives that may or may not be part of the skin patch; inhalers for dispensing therapeutic agents in an inhalable form such as, for example, those disclosed in U.S. Pat. No. 6,354,290 and U.S. Pat. No. 6,364,865, among others; and a suitable diluent for dissolving a solid composition, among other possible devices or supplies that may be included in such a kit.

Compositions comprising therapeutic molecules described above can be administered by any appropriate means including, but not limited to, parenteral, topical, oral, nasal, vaginal, rectal, or pulmonary (by inhalation) administration. If injected, the composition(s) can be administered intra-articularly, intravenously, intraarterially, intramuscularly, intralesionally, intraperitoneally, or subcutaneously by bolus injection or continuous infusion. Localized administration, that is, at the site of disease, is contemplated, as are transdermal delivery and sustained release from implants or skin patches. Delivery by inhalation includes, for example, nasal or oral inhalation, use of a nebulizer, inhalation in aerosol form, and the like. Administration via a suppository inserted into a body cavity can be accomplished, for example, by inserting a solid form of the composition in a chosen body cavity and allowing it to dissolve. Other alternatives include eyedrops, oral preparations such as pills, lozenges, syrups, and chewing gum, and topical preparations such as lotions, gels, sprays, and ointments. In most cases, therapeutic molecules that are polypeptides can be administered topically or by injection or inhalation. Small molecules can, in most cases, be administered orally.

The therapeutic molecules described above can be administered at any dosage, frequency, and duration that can be effective to treat the condition being treated. The dosage depends on the molecular nature of the EGFR inhibitor and the nature of the disorder being treated. Treatment may be continued as long as necessary to achieve the desired results. EGFR inhibitors can be administered as a single dosage or as a series of dosages given periodically, including multiple times per day, daily, every other day, twice a week, three times per week, weekly, every other week, and monthly dosages, among other possible dosage regimens. The periodicity of treatment may or may not be constant throughout the duration of the treatment. For example, treatment may initially occur at weekly intervals and later occur every other week. Treatments having durations of days, weeks, months, or years are encompassed by the invention. Treatment may be discontinued and then restarted. Maintenance doses may be administered after an initial treatment.

Dosage may be measured as milligrams per kilogram of body weight (mg/kg) or as milligrams per square meter of skin surface (mg/m²) or as a fixed dose, irrespective of height or weight. All of these are standard dosage units in the art. A person's skin surface area is calculated from her height and weight using a standard formula. With respect to EGFR inhibitors that are antibodies, dosages can range from about 0.01 mg/kg to about 70 mg/kg, optionally from about 0.1 mg/kg to about 20 mg/kg, from about 0.1 mg/kg to about 5 mg/kg, from about 0.3 mg/kg to about 3 mg/kg, or about 2.5 mg/kg. Alternatively, patients of all sizes can receive the same dosage, ranging from about 1 mg to about 500 mg, optionally from about 10 mg to about 100 mg or from about 25 mg to about 50 mg. Alternatively, the dosage may be from about 5 mg/m to about 800 mg/m², from about 10 mg/m² to about 600 mg/m², or from about 25 mg/m to about 500 mg/m². Dosages may or may not be constant throughout the duration of the treatment. For example, dosage may steadily escalate throughout the duration of the treatment. Alternatively, a first dose may be higher than subsequent doses. As a further alternative, dosage may be reduced at later stages of the treatment. As an illustration, the ABX-EGF antibody (described above) can be administered in, for example, doses from about 0.1 mg/kg to about 5 mg/kg, preferably about 2.5 mg/kg. Such doses can be administered, for example, at intervals of once a week or once every two weeks. As another illustration, the C225 antibody (described above) can be administered by infusion with a first dose of from about 100 mg/m² to about 500 mg/m², and subsequent weekly doses of from about 100 mg/m² to about 250 mg/m².

Dosages for polypeptide EGFR inhibitors other than antibodies may be different than those for antibodies. For example, VRCTC-310 dosages can range from about 0.001 mg/kg to about 6.3 mg/kg daily, optionally from about 0.010 mg/kg to about 1.0 mg/kg or from about 0.014 mg/kg to about 0.017 mg/kg at from daily to weekly intervals. The cyclic peptides described in U.S. Pat. No. 5,183,805 may be administered in doses from about 0.1 mg/kg daily to about 200 mg/kg daily.

With respect to EGFR inhibitors that are small molecules, optimal dosages will depend on the nature of the small molecule as well as the condition being treated. In most cases, small molecule EGFR inhibitors can be administered orally. One of skill in the art can determine an optimal dosage using guidance provided herein as well as the knowledge within the art. For example, OSI-774 can be administered as a single agent in oral doses from about 5 mg/kg to about 250 mg/kg daily, optionally at doses from about 25 mg/kg to about 150 mg/kg at from daily to weekly intervals. C₁₋₁₀₃₃ can be administered as a single agent in oral doses from about 5 mg/kg to about 800 mg/kg, optionally from about 50 mg/kg to about 560 mg/kg at intervals from daily to every other week. ZD-1839 can be administered as a single agent orally in doses from about 5 mg/kg to about 1100 mg/kg, optionally from about 50 mg/kg to about 700 mg/kg, including doses of 50 mg/kg, 100 mg/kg, 225 mg/kg, 250 mg/kg, and 500 mg/kg at from daily to weekly intervals.

One of skill in the art will recognize that doses of each therapeutic agent in a combination of an EGFR inhibitor and another therapeutic agent may be adjusted to achieve the desired response. Doses appropriate for the use of either or both therapeutic agents as part of a combination may be lower than doses appropriate for use as a single agent.

The foregoing description of the specific embodiments reveals the general nature of the invention so that others can readily modify and/or adapt such embodiments for various applications without departing from the generic concepts presented herein. Any such adaptions or modifications are intended to be embraced within the meaning and range of equivalents of the disclosed embodiments. Phraseology and terminology employed herein are for the purpose of description and not of limitation. All references cited herein are incorporated by reference in their entirety.

What is claimed is:

1. A method for reducing tumor burden comprising administering a therapeutically effective amount of an EGFR inhibitor to a human patient suffering from a cancer in which the cancer cells express the CCK_{B}/gastrin receptor and express little or no EGFR.

2. The method of claim 1, wherein the EGFR inhibitor is a small molecule.

3. A method for inhibiting recurrence of gross cystic disease of the breast and/or inhibiting the progression from gross cystic disease of the breast to breast cancer comprising administering to a patient who presently has or has had gross cystic disease of the breast a therapeutically effective amount of an EGFR inhibitor.

4. The method of claim 3, wherein the EGFR inhibitor is administered orally.

5. A method for preventing or reducing the frequency or severity of transient ischemic attacks or strokes comprising administering to a patient suffering from cerebral ischemia a therapeutically effective amount of an EGFR inhibitor.

6. The method of claim 5, wherein the patient suffers from hypertension.

7. The method of claim 5, wherein the patient suffers from atherosclerosis.
